(19) United States
(12) Patent Application Publication
(10) Pub. No.: US 2003/0096813 A1
(43) Pub. Date: May 22, 2003

(54) Compositions useful as inhibitors of GSK-3

(76) Inventors: Jingrong Cao, Newton, MA (US); Debbie Choquette, Medford, MA (US); Robert Davies, Arlington, MA (US); Cornelia Forster, Pelham, NH (US); David Lauffer, Stow, MA (US); Albert Pierce, Somerville, MA (US); Ronald Tomlinson, Marlborough, MA (US); Marion Wannamaker, Stow, MA (US); Natalie Metz, Brighton, MA (US)

Correspondence Address: VERTEX PHARMACEUTICALS INCORPORATED, 130 Waverly Street, Cambridge, MA, 02130-4646, US

(21) Appl. No.: 10/125,885

(22) Filed: Apr. 19, 2002

Related U.S. Application Data
(60) Provisional application No. 60/285,217, filed on Apr. 20, 2001.

(51) Int. Cl. A61K 31/541; A61K 31/5377; A61K 31/519; C07D 498/02; C07D 487/02

(52) U.S. Cl. 514/228.5; 514/234.5; 514/252.16; 514/260.1; 514/265.1; 544/60; 544/117; 544/278; 544/280

(57) ABSTRACT

The present invention provides compounds of formula I:

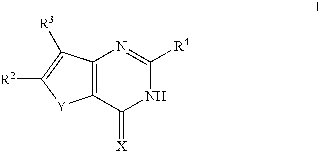

or a pharmaceutically acceptable derivative thereof, wherein X is oxygen or sulfur; Y is —S—, —O—, or —NR¹—; and R², R³, and R⁴ are as described in the specification. These compounds are inhibitors of protein kinase, particularly inhibitors of GSK-3 mammalian protein kinase. The invention also provides pharmaceutical compositions comprising the inhibitors of the invention and methods of utilizing those compositions in the treatment and prevention of various disorders, such as diabetes and Alzheimer's disease.

### CROSS REFERENCE TO RELATED APPLICATIONS

This application claims priority to U.S. Provisional Patent Application 60/205,217 filed Apr. 20, 2001, the contents of which are incorporated herein by reference.

### FIELD OF THE INVENTION

The present invention is in the field of medicinal chemistry and relates to compounds that are protein kinase inhibitors, compositions containing such compounds and methods of use. More particularly, the compounds are inhibitors of GSK-3 and are useful for treating or lessening the severity of diseases or conditions, such as diabetes and Alzheimer's disease, that are alleviated by GSK-3 inhibitors.

### BACKGROUND OF THE INVENTION

The search for new therapeutic agents has been greatly aided in recent years by better understanding of the structure of enzymes and other biomolecules associated with target diseases. One important class of enzymes that has been the subject of extensive study is the protein kinases.

Protein kinases mediate intracellular signal transduction. They do this by effecting a phosphoryl transfer from a nucleoside triphosphate to a protein acceptor that is involved in a signaling pathway. There are a number of kinases and pathways through which extracellular and other stimuli cause a variety of cellular responses to occur inside the cell. Examples of such stimuli include environmental and chemical stress signals (e.g. osmotic shock, heat shock, ultraviolet radiation, bacterial endotoxin, H₂O₂), cytokines (e.g. interleukin-1 (IL-1) and tumor necrosis factor α (TNF-α)), and growth factors (e.g. granulocyte macrophage-colony-stimulating factor (GM-CSF), and fibroblast growth factor (FGF). An extracellular stimulus may effect one or more cellular responses related to cell growth, migration, differentiation, secretion of hormones, activation of transcription factors, muscle contraction, glucose metabolism, control of protein synthesis and regulation of cell cycle.

Many disease states are associated with abnormal cellular responses triggered by protein kinase-mediated events. These diseases include autoimmune diseases, inflammatory diseases, metabolic diseases, neurological and neurodegenerative diseases, cancer, cardiovascular diseases, allergies and asthma, Alzheimer's disease or hormone-related diseases. Accordingly, there has been a substantial effort in medicinal chemistry to find protein kinase inhibitors that are effective as therapeutic agents.

Glycogen synthase kinase-3 (GSK-3) is a serine/threonine protein kinase comprised of α and β isoforms that are each encoded by distinct genes [Coghlan et al., Chemistry & Biology, 7, 793-803 (2000); Kim and Kimmel, Curr. Opinion Genetics Dev., 10, 508-514 (2000)]. GSK-3 has been implicated in various diseases including diabetes, Alzheimer's disease, CNS disorders such as manic depressive disorder and neurodegenerative diseases, and cardiomyocete hypertrophy [WO 99/65897; WO 00/38675; and Haq et al., J. Cell Biol. (2000) 151, 117]. These diseases may be caused by, or result in, the abnormal operation of certain cell signaling pathways in which GSK-3 plays a role. GSK-3 has been found to phosphorylate and modulate the activity of a number of regulatory proteins. These include glycogen synthase which is the rate limiting enzyme necessary for glycogen synthesis, the microtubule associated protein Tau, the gene transcription factor β-catenin, the translation initiation factor e1F2B, as well as ATP citrate lyase, axin, heat shock factor-1, c-Jun, c-Myc, c-Myb, CREB, and CEPBα. These diverse targets implicate GSK-3 in many aspects of cellular metabolism, proliferation, differentiation and development.

In a GSK-3 mediated pathway that is relevant for the treatment of type II diabetes, insulin-induced signaling leads to cellular glucose uptake and glycogen synthesis. Along this pathway, GSK-3 is a negative regulator of the insulin-induced signal. Normally, the presence of insulin causes inhibition of GSK-3 mediated phosphorylation and deactivation of glycogen synthase. The inhibition of GSK-3 leads to increased glycogen synthesis and glucose uptake [Klein et al., PNAS, 93, 8455-9 (1996); Cross et al., Biochem. J., 303, 21-26 (1994); Cohen, Biochem. Soc. Trans., 21, 555-567 (1993); Massillon et al., Biochem J. 299, 123-128 (1994)]. However, in a diabetic patient where the insulin response is impaired, glycogen synthesis and glucose uptake fail to increase despite the presence of relatively high blood levels of insulin. This leads to abnormally high blood levels of glucose with acute and chronic effects that may ultimately result in cardiovascular disease, renal failure and blindness. In such patients, the normal insulin-induced inhibition of GSK-3 fails to occur. It has also been reported that in patients with type II diabetes, GSK-3 is over expressed [WO 00/38675]. Therapeutic inhibitors of GSK-3 are therefore potentially useful for treating diabetic patients suffering from an impaired response to insulin.

GSK-3 activity has also been associated with Alzheimer's disease. This disease is characterized by the well-known β-amyloid peptide and the formation of intracellular neurofibrillary tangles. The neurofibrillary tangles contain hyperphosphorylated Tau protein where Tau is phosphorylated on abnormal sites. GSK-3 has been shown to phosphorylate these abnormal sites in cell and animal models. Furthermore, inhibition of GSK-3 has been shown to prevent hyperphosphorylation of Tau in cells [Lovestone et al., Current Biology 4, 1077-86 (1994); Brownlees et al., Neuroreport 8, 3251-55 (1997)]. Therefore, it is believed that GSK-3 activity may promote generation of the neurofibrillary tangles and the progression of Alzheimer's disease.

Another substrate of GSK-3 is β-catenin which is degradated after phosphorylation by GSK-3. Reduced levels of β-catenin have been reported in schizophrenic patients and have also been associated with other diseases related to increase in neuronal cell death [Zhong et al., Nature, 395, 698-702 (1998); Takashima et al., PNAS, 90, 7789-93 (1993); Pei et al., J. Neuropathol. Exp, 56, 70-78 (1997); Smith et al., Bio-org. Med. Chem. 11, 635-639 (2001)]. Recently, GSK-3 inhibition has been shown to prevent neuronal cell death in vitro and has been implicated in the neuronal cell death pathway caused by ischemic stress (Cross et al, J.Neurochemistry, 2001, 77, 94-102; Sasaki et al, Neurological Research, 2001, 23,588-592) implicating GSK-3 as a target in the treatment of stroke.

Small molecule inhibitors of GSK-3 have recently been reported [WO 99/65897 (Chiron) and WO 00/38675 (SmithKline Beecham)].

Another kinase of interest is Rho-associated coiled-coil forming kinase (ROCK) [Ishizaki et al., EMBO J. 1996, 15, 1885-1893]. ROCK kinase is a 160 kDa serine/threonine kinase that activates the small G-protein RhoA. ROCK has been implicated in numerous diseases including hypertension [Chitaley et al. Curr Hypertens Rep 2001 April;3(2):139-44; Uehata et al., Nature, 1997, 389, 990-994], erectile dysfunction [Chitaley et al. Nature Medicine, 2001, 7, 119-122], angiogenesis [Uchida et al., Biochem Biophys Res Commun 2000, 269 (2), 633-40], neuroregeneration [Bito et al., Neuron, 2000, 26, 431-441], metastasis [Takamura et al., Hepatology, 2001, 33, 577-581; Genda et al., Hepatology, 1999, 30, 1027-1036], glaucoma [Rao et al., Invest Ophthalmol Vis Sci 2001, 42, 1029-37], inflammation [Ishizuki et al., J. Immunol., 2001, 167, 2298-2304], artheriosclerosis [Smimokawa et al., Arterioscler. Thromb. Vasc. Biol., 2000, 11, 2351-2358], immunosuppresion [Lou et al., J. Immunol., 2001, 167, 5749-5757], restenosis [Seaholtz et al., Circ. Res., 1999, 84, 1186-1193], asthma [Yoshii et al., Am. J. Respir. Cell Mol. Biol., 1999, 20, 1190-1200], and cardiac hypertrophy [Kuwahara et al., FEBS Lett., 1999, 452, 314-318].

There is a continued need to find new therapeutic agents to treat human diseases. The protein kinase GSK-3, in particular GSK-3β, and ROCK kinase are especially attractive targets for the discovery of new therapeutics due to their important role in diabetes, Alzheimer's disease, and various other diseases.

### DESCRIPTION OF THE INVENTION

It has now been found that compounds of this invention, and pharmaceutically acceptable compositions comprising said compounds, are effective as protein kinase inhibitors, particularly as inhibitors of GSK-3. Accordingly, the present invention relates to a compound of formula I:

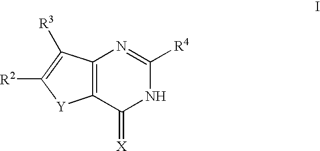

or a pharmaceutically acceptable derivative thereof, wherein:

X is oxygen or sulfur;

Y is —S—, —O—, or —NR¹—;

R¹ is selected from R, CO₂R, C(O)R, CON(R)₂, SO₂R, SO₂N(R)₂, or an optionally substituted 5-7 membered monocyclic or 8-10 membered bicyclic saturated, partially unsaturated, or fully unsaturated ring having 0-3 heteroatoms independently selected from nitrogen, oxygen, or sulfur;

each R is independently selected from hydrogen or an optionally substituted C₁₋₆ aliphatic group;

R² is selected from R, N(R)₂, OR, SR, C(O)R, CO₂R, C(O)N(R)₂, NRN(R)₂, NRCOR, NRCO₂(C₁₋₆ aliphatic), NRSO₂(C₁₋₆ aliphatic), S(O)(C₁₋₆aliphatic), SO₂R, SO₂N(R)₂, or an optionally substituted 5-7 membered monocyclic or 8-10 membered bicyclic saturated, partially unsaturated, or fully unsaturated ring having 0-3 heteroatoms independently selected from nitrogen, oxygen, or sulfur, or:

(a) when Y is —NR¹, R¹ and R² are taken together to form a saturated, partially unsaturated, or fully unsaturated 4-9 membered mono- or bicyclic ring having 1-2 heteroatoms, in addition to the —NR¹— nitrogen, independently selected from nitrogen, oxygen, or sulfur, wherein said ring formed by R¹ and R² is optionally substituted with 1-2 R⁶; or

(b) R² and R³ are taken together to form a saturated, partially unsaturated, or fully unsaturated 5-9 membered mono- or bicyclic ring having 0-2 heteroatoms independently selected from nitrogen, oxygen, or sulfur, wherein said ring formed by R² and R³ is optionally substituted with 1-2 R⁶;

R³ is selected from R, CN, halogen, NO₂, or Q₍ₙ₎R⁵, wherein:

n is selected from zero or one;

Q is a C₁₋₄ straight or branched alkylidene chain, wherein up to two non-adjacent methylene units of Q are optionally and independently replaced by O, S, NR, C(O), CO₂, CONR, OC(O)NR, NRCO, NRCO₂, NRCONR, S(O), SO₂, NRSO₂, or SO₂NR;

R⁴ is selected from R, N(R)₂, NRCOR, NRCO₂R, or an optionally substituted 5-7 membered monocyclic or 8-10 membered bicyclic saturated, partially unsaturated, or fully unsaturated ring having 0-3 heteroatoms independently selected from nitrogen, oxygen, or sulfur;

R⁵ is selected from R or an optionally substituted 5-14 membered mono-, bi-, or tricyclic aromatic, partially unsaturated, or saturated ring having 0-4 heteroatoms independently selected from nitrogen, oxygen, or sulfur; and

each R⁶ is independently selected from R, oxo, halogen, CN, C(O)R, CO₂R, SO₂R, OR, SR, N(R)₂, NRC(O)R, C(O)N(R)₂, NRCO₂R, OC(O)N(R)₂, NRSO₂R, or SO₂NR.

As used herein, the following definitions shall apply unless otherwise indicated.

The term “optionally substituted” is used interchangeably with the term “substituted or unsubstituted.” Each of those terms refers to the possibility, but not the requirement, that one or more hydrogen atoms are replaced by another moiety. When an optional substituent includes hydrogen within its definition, it should be understood that hydrogen is specifically excluded as a choice for such substitution.

The term “aliphatic” or “aliphatic group” as used herein means a straight-chain or branched C₁-C₁₂ hydrocarbon chain that is completely saturated or that contains one or more units of unsaturation, or a monocyclic C₃-C₈ hydrocarbon or bicyclic C₈-C₁₂ hydrocarbon that is completely saturated or that contains one or more units of unsaturation, but which is not aromatic (also referred to herein as “carbocycle” or “cycloalkyl”), that has a single point of attachment to the rest of the molecule wherein any individual ring in said bicyclic ring has three to seven members. For example, suitable aliphatic groups include, but are not limited to, linear or branched or alkyl, alkenyl, alkynyl groups and hybrids thereof such as (cycloalkyl)alkyl, (cycloalkenyl)alkyl or (cycloalkyl)alkenyl.

The terms “alkyl”, “alkenyl” and “alkynyl” used alone or as part of a larger moiety shall include both straight and branched chains containing one to twelve carbon atoms and at least two carbon atoms and one double bond in the case of alkenyl and at least two carbon atoms and one triple bond, in the case of alkynyl.

The term “alkylidene chain” refers to a straight or branched carbon chain that may be fully saturated or have one or more units of unsaturation and has two points of attachment to the rest of the molecule.

The terms “halo” and “halogen” used alone or as part of a larger moiety means F, Cl, Br, or I.

The term “methylene group” or “-methylene unit-” refers to any —CH₂— moiety present in an aliphatic or alkylidene, including the —CH₂— portion of a terminal —CH₃ group in an aliphatic.

The term “heteroatom” means nitrogen, oxygen, or sulfur and includes any oxidized form of nitrogen and sulfur, and the quaternized form of any basic nitrogen.

The term “aryl”, used alone or as part of a larger moiety as in “aralkyl”, refers to monocyclic, bicyclic and tricyclic ring systems having a total of five to fourteen ring members, wherein at least one ring in the system is aromatic and wherein each ring in the system contains three to seven ring members. The term “aryl” may be used interchangeably with the term “aryl ring”. The term “aryl” also refers to “heteroaryl” rings.

The term “heteroaryl”, used alone or as part of a larger moiety as in “heteroaralkyl” or “heteroarylalkoxy”, refers to monocyclic, bicyclic and tricyclic ring systems having a total of five to fourteen ring members, wherein at least one ring in the system is aromatic, at least one ring in the system contains one or more heteroatoms, and wherein each ring in the system contains 3 to 7 ring members. The term “heteroaryl” may be used interchangeably with the term “heteroaryl ring” or the term “heteroaromatic”.

The terms aryl and heteroaryl include rings such as phenyl, benzyl, 1-naphthyl, 2-naphthyl, 1-anthracyl and 2-anthracyl, 2-furanyl, 3-furanyl, N-imidazolyl, 2-imidazolyl, 4-imidazolyl, 5-imidazolyl, 3-isoxazolyl, 4-isoxazolyl, 5-isoxazolyl, 2-oxadiazolyl, 5-oxadiazolyl, 2-oxazolyl, 4-oxazolyl, 5-oxazolyl, 2-pyrrolyl, 3-pyrrolyl, 2-pyridyl, 3-pyridyl, 4-pyridyl, 2-pyrimidyl, 4-pyrimidyl, 5-pyrimidyl, 3-pyridazinyl, 2-thiazolyl, 4-thiazolyl, 5-thiazolyl, 5-tetrazolyl, 2-triazolyl, 5-triazolyl, 2-thienyl, or 3-thienyl.

Examples of fused polycyclic aromatic ring systems in which a carbocyclic aromatic ring or heteroaryl ring is fused to one or more other rings include tetrahydronaphthyl, benzimidazolyl, benzothienyl, benzofuranyl, indolyl, quinolinyl, benzothiazolyl, benzoxazolyl, benzimidazolyl, isoquinolinyl, isoindolyl, acridinyl, benzoisoxazolyl, and the like. Also included within the scope of the term “aryl”, as it is used herein, is a group in which one or more carbocyclic aromatic rings and/or heteroaryl rings are fused to a cycloalkyl or non-aromatic heterocyclic ring, for example, indanyl, 1-phthalimidinyl, benzoxane, benzotriazol-1-yl, benzopyrrolidine, benzopiperidine, benzoxolane, benzothiolane, benzothiane, or tetrahydrobenzopyranyl.

The term “heterocycle”, “heterocyclyl”, or “heterocyclic” as used herein means non-aromatic, monocyclic, bicyclic or tricyclic ring systems having five to fourteen ring members in which one or more ring members is a heteroatom, wherein each ring in the system contains three to seven ring members. Examples include 3-1H-benzimidazol-2-one, 3-1H-alkyl-benzimidazol-2-one, 2-tetrahydrofuranyl, 3-tetrahydrofuranyl, 2-tetrahydrothiophenyl, 3-tetrahydrothiophenyl, 2-morpholino, 3-morpholino, 4-morpholino, 2-thiomorpholino, 3-thiomorpholino, 4-thiomorpholino, 1-pyrrolidinyl, 2-pyrrolidinyl, 3-pyrrolidinyl, 1-piperazinyl, 2-piperazinyl, 1-piperidinyl, 2-piperidinyl, 3-piperidinyl, 4-piperidinyl, 4-thiazolidinyl, diazolonyl, and N-substituted diazolonyl.

An aryl (including aralkyl, aralkoxy, aryloxyalkyl and the like) or heteroaryl (including heteroaralkyl and heteroarylalkoxy and the like) group may contain one or more substituents. Suitable substituents on the unsaturated carbon atom of an aryl, heteroaryl, aralkyl, or heteroaralkyl group are selected from halogen, —R⁰, —OR⁰, —SO⁰, 1,2-methylene-dioxy, 1,2-ethylenedioxy, phenyl (Ph) optionally substituted with R⁰, —O(Ph) optionally substituted with R⁰, —CH₂(Ph) optionally substituted with R⁰, —CH₂CH₂(Ph) optionally substituted with R⁰, —NO₂, —CN, —N(R⁰)₂, —NR⁰C(O)R⁰, —NR⁰C(O)N(R⁰)₂, —NR⁰CO₂R⁰, —NR⁰NR⁰C(O)R⁰, —NR⁰NR⁰C(O)N(R⁰)₂, —NR⁰NR⁰CO₂R⁰, —C(O)C(O)R⁰, —C(O)CH₂C(O)R⁰, —CO₂R⁰, —C(O)R⁰, —C(O)N(R⁰)₂, —OC(O)N(R⁰)₂, —S(O)₂R⁰, —SO₂N(R⁰)₂, —S(O)R⁰, —NR⁰SO₂N(R⁰)₂, —NR⁰SO₂R⁰, —C(═S)N(R⁰)₂, —C(═NH)—N(R⁰)₂, or —(CH₂)_{y}NHC(O)R⁰, wherein each R⁰ is independently selected from hydrogen, optionally substituted C₁₋₆ aliphatic, an unsubstituted 5-6 membered heteroaryl or heterocyclic ring, phenyl, —O(Ph), or —CH₂(Ph). Optional substituents on the aliphatic group of R⁰ are selected from NH₂, NH(C₁₋₄ aliphatic), N(C₁₋₄ aliphatic)₂, halogen, C₁₋₄ aliphatic, OH, O(C₁₋₄ aliphatic), NO₂, CN, CO₂H, CO₂(C₁₋₄ aliphatic), O(halo C₁₋₄ aliphatic), or halo C₁₋₄ aliphatic, wherein each C₁₋₄ aliphatic group is unsubstituted.

An aliphatic group or a non-aromatic heterocyclic ring may contain one or more substituents. Suitable substituents on the saturated carbon of an aliphatic group or of a non-aromatic heterocyclic ring are selected from those listed above for the unsaturated carbon of an aryl or heteroaryl group and the following: ═O, ═S, ═NNHR*, ═NN (R*)₂, ═NNHC(O)R*, ═NNHCO₂(alkyl), ═NNHSO₂(alkyl), or ═NR*, where each R* is independently selected from hydrogen or an optionally substituted C₁₋₆ aliphatic. Optional substituents on the aliphatic group of R* are selected from NH₂, NH(C₁₋₄ aliphatic), N(C₁₋₄ aliphatic)₂, halogen, C₁₋₄ aliphatic, OH, O(C₁₋₄ aliphatic), NO₂, CN, CO₂H, CO₂(C₁₋₄ aliphatic), O(halo C₁₋₄ aliphatic), or halo(C₁₋₄ aliphatic), wherein each C₁₋₄ aliphatic group is unsubstituted.

Optional substituents on the nitrogen of a non-aromatic heterocyclic ring are selected from —R⁺, —N(R⁺)₂, —C(O)R⁺, —CO₂R⁺, —C(O)C(O)R⁺, —C(O)CH₂C(O)R+, —SO₂R⁺, —SO₂N(R⁺)₂, —C(═S)N(R⁺)₂, —C(═NH)—N(R⁺)₂, or —NR ⁺SO₂R⁺; wherein R⁺ is hydrogen, an optionally substituted C₁₋₆ aliphatic, optionally substituted phenyl, optionally substituted —O(Ph), optionally substituted —CH₂(Ph), optionally substituted —CH₂CH₂(Ph), or an unsubstituted 5-6 membered heteroaryl or heterocyclic ring. Optional substituents on the aliphatic group or the phenyl ring of R⁺ are selected from NH₂, NH(C₁₋₄ aliphatic), N(C₁₋₄ aliphatic)₂, halogen, C₁₋₄ aliphatic, OH, O(C₁₋₄ aliphatic), NO₂, CN, CO₂H, CO₂(C₁₋₄ aliphatic), O(halo C₁₋₄ aliphatic), or halo(C₁₋₄ aliphatic), wherein each C₁₋₄ aliphatic group is unsubstituted.

A combination of substituents or variables is permissible only if such a combination results in a stable or chemically feasible compound. A stable compound or chemically feasible compound is one that is not substantially altered when kept at a temperature of 40° C. or less, in the absence of moisture or other chemically reactive conditions, for at least a week.

It will be apparent to one skilled in the art that certain compounds of this invention may exist in tautomeric forms, all such tautomeric forms of the compounds being within the scope of the invention.

Unless otherwise stated, structures depicted herein are also meant to include all stereochemical forms of the structure; i.e., the R and S configurations for each asymmetric center. Therefore, single stereochemical isomers as well as enantiomeric and diastereomeric mixtures of the present compounds are within the scope of the invention. Unless otherwise stated, structures depicted herein are also meant to include compounds that differ only in the presence of one or more isotopically enriched atoms. For example, compounds having the present structures except for the replacement of a hydrogen by a deuterium or tritium, or the replacement of a carbon by a ¹³C- or ¹⁴C-enriched carbon are within the scope of this invention.

Preferred R¹ groups of formula I are selected from R, C(O)R, C(O)N(R)₂, SO₂R, CO₂R, or an optionally substituted 5-6 membered saturated, partially unsaturated, or fully unsaturated ring having 0-2 heteroatoms independently selected from nitrogen, oxygen, or sulfur, wherein each R is as defined above. More preferred R¹ groups of formula I are selected from hydrogen, methyl, ethyl, i-propyl, i-butyl, phenyl, CH₂CH₂(morpholin-4-yl), CH₂CH₂phenyl, CH₂phenyl, COMe, CONH₂, CH₂CONH₂, SO₂Me, CH₂SO₂NH₂, CO₂Et, or cyclopropyl.

Preferred R² groups of formula I are selected from R, N(R)₂, OR, SR, C(O)R, CO₂R, C(O)N(R)₂, NRN(R)₂, NRC(O)R, SO₂R, or an optionally substituted 5-7 membered saturated, partially unsaturated, or fully unsaturated ring having 0-2 heteroatoms independently selected from nitrogen, oxygen, or sulfur. More preferred R² groups of formula I are selected from hydrogen, methyl, ethyl, i-propyl, i-butyl, CF₃, phenyl, CH₂CH₂NH₂, NH₂, NHC(O)CH₃, CH₂CH₂NHC(O)OCH₂phenyl, SCH₃, SO₂CH₃, NHCH₃, SEt, CH₂phenyl, Oi-propyl, morpholin-4-yl, piperidin-1-yl, 4-methyl-piperazin-1-yl, thiomorpholin-4-yl, pyrrolidin-1-yl, thiazol-3-yl, oxazol-3-yl, azepan-1-yl, N(Me)₂, NHi-propyl, NHpropyl, NHi-butyl, NH-cyclopentyl, NH-cyclohexyl, NHCH₂phenyl, NHSO₂CH₃, NHNH₂, N(Me)propyl, NH-cyclopropyl, NHCH₂cyclohexyl, NHCH₂CH₂CH (CH₃)₂, or NHCH₂CH₂imidazol-4-yl.

When Y is —NR¹— and R² and R¹ are taken together to form a ring, preferred rings formed by R² and R¹ are selected from an optionally substituted 5-8 membered saturated, partially unsaturated, or aromatic ring having 0-2 heteroatoms, in addition to the nitrogen of R¹, independently selected from nitrogen, oxygen, or sulfur. More preferred rings formed by R² and R¹ are selected from a cyclopento, cyclohexo, cyclohepto, benzo, pyrido, pyridazo, oxacyclohepto, tetrahydroazepino, or thiacyclohepto ring. When the ring formed by R² and R¹ is substituted by R⁶, preferred R⁶ substituents are selected from R, OR, N(R)₂, oxo, halogen, NRCO₂R, or NRC(O)R. More preferred R₆ groups are NH₂, methyl, OCH₃, NHCOCH₃, NHCO₂CH₃, or N(Me)₂.

Preferred R³ groups of formula I are selected from R, CN, or Q₍ₙ₎R⁵₁ wherein n is zero or one, Q is selected from a C₁₋₄ alkylidene chain wherein one methylene unit of Q is optionally replaced by O, S, NR, C(O), CO₂, CONR, NRC(O), NRC(O)NR, SO₂, or NRSO₂, and R⁵ is selected from R or an optionally substituted 5-7 membered saturated, partially unsaturated, or fully unsaturated ring having 0-4 heteroatoms independently selected from nitrogen, oxygen, or sulfur. More preferred R³ groups of formula I are selected from hydrogen, CN, CO₂H, CH₂CN, methyl, CH₂CONH₂, CH₂CO₂CH₃, —C═CH, C(O)CH₃, CH₂CH₂CN, CH₂CH₂CH₂NH₂, hydrogen, CH₂CO₂H, CO₂Et, CH₂SO₂CH₃, CH₂NHSO₂CH₃, C(O)NH₂, CH₂NHC(O)CH₃, CH₂CH₂OH, C(O)CH₂CH₃, oxadiazolyl, NH₂, NHC(O)CH₃, NHSO₂CH₃, NHCO₂CH₃, tetrazolyl, C(O)piperidin-1-yl, C(O)morpholin-4-yl, C(O)thiomorpholin-4-yl, C(O)-4-methylpiperazin-1-yl, C(O)NHCH₂phenyl, CH₂NHCONH₂, CH₂NHS)₂phenyl, triazolyl, thiadiazolyl, thiazolyl, oxazolyl, pyrazolyl, isoxazolyl, C(O)NH-thiazol-2-yl, C(O)NH-pyrazol-3-yl, or C(O)NHC(CH₃)₃.

Preferred R⁴ groups of formula I are selected from R, N(R)₂, or an optionally substituted 5-6 membered saturated, partially unsaturated, or fully unsaturated ring having 0-2 heteroatoms independently selected from nitrogen, oxygen, or sulfur. More preferred R⁴ groups of formula I are selected from hydrogen, methyl, ethyl, propyl, i-propyl, cyclopropyl, CF₃, phenyl, NH₂, CH₂phenyl, or N(CH₃)CH₂phenyl.

One embodiment of this invention relates to compounds of formula I where Y is —NR¹—, represented by formula II:

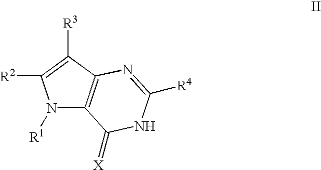

or a pharmaceutically acceptable derivative thereof, wherein R¹, R², R³, R⁴, and X are as defined above for formula I.

Preferred R¹, R^{2,} R³, and R⁴ groups for formula II are those described above for compounds of formula I.

More preferred compounds of formula II have one or more, and more preferably all, of the features selected from the group consisting of:

(a) R¹ is selected from R, C(O)R, C(O)N(R)₂, SO₂R, CO₂R, or an optionally substituted 5-6 membered saturated, partially unsaturated, or fully unsaturated ring having 0-2 heteroatoms independently selected from nitrogen, oxygen, or sulfur;

(b) R² is selected from R, N(R)₂, OR, SR, C(O)R, CO₂R, C(O)N(R)₂, NRN(R)₂, NRC(O)R, SO₂R, or an optionally substituted 5-7 membered saturated, partially unsaturated, or fully unsaturated ring having 0-2 heteroatoms independently selected from nitrogen, oxygen, or sulfur, or R² and R¹ are taken together to form an optionally substituted 5-8 membered saturated, partially unsaturated, or aromatic ring having 0-1 heteroatoms, in addition to the nitrogen of R¹, independently selected from nitrogen, oxygen, or sulfur;

(c) R³ is selected from R, CN, or Q₍ₙ₎R⁵, wherein n is zero or one, Q is selected from a C₁₋₄ alkylidene chain wherein one methylene unit of Q is optionally replaced by O, S, NR, C(O), CO₂, CONR, NRC(O), NRC(O)NR, SO₂, or NRSO₂, and R⁵ is selected from R or an optionally substituted 5-7 membered saturated, partially unsaturated, or fully unsaturated ring having 0-4 heteroatoms independently selected from nitrogen, oxygen, or sulfur; and

(d) R⁴ is selected from R, N(R)₂, or an optionally substituted 5-6 membered saturated, partially unsaturated, or fully unsaturated ring having 0-2 heteroatoms independently selected from nitrogen, oxygen, or sulfur.

One aspect of this embodiment relates to compounds of formula II where R¹ and R² are taken together to form a ring. Compounds of formula II where the ring formed by R¹ and R² contains one heteroatom, the nitrogen to which R¹ is attached, are represented by formula II-A:

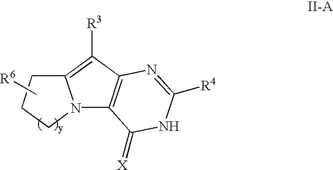

or a pharmaceutically acceptable derivative thereof, wherein y is 0-4 and R³, R⁴, X, and R⁶ are as defined above.

Preferred R³, R⁴, X, and R⁶ groups of formula II-A are those described above for compounds of formula I. The ring formed by R¹ and R² is preferably a 5-8 membered ring (y is 1-4).

Representative examples of compounds of formula II-A are shown below in Table 1.

TABLE 1
Examples of Compounds II-A

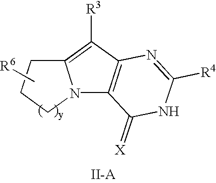

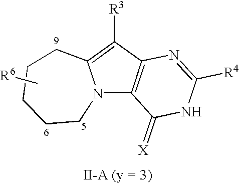

| No. | y | X | R³ | R⁴ | R⁶ |
|---|---|---|---|---|---|
| II-A1 | 1 | S | —CN | H | H |
| II-A2 | 2 | S | —CN | H | H |
| II-A3 | 3 | S | —CN | H | H |
| II-A4 | 4 | S | —CN | H | H |
| II-A5 | 3 | S | —CO₂H | H | H |
| II-A6 | 3 | S | —CH₂CN | H | H |
| II-A7 | 3 | S | —CH₃ | H | H |
| II-A8 | 3 | S | —CH₂CONH₂ | H | H |
| II-A9 | 3 | S | —CH₂CO₂CH₃ | H | H |
| II-A10 | 3 | S | —C≡CH | H | H |
| II-A11 | 3 | S | —COCH₃ | H | H |
| II-A12 | 3 | S | —C(CH₃)═N—OCH₃ | H | H |
| II-A13 | 3 | S | —CH₂CH₂CN | H | H |
| II-A14 | 3 | S | —C(CH₃)═NNHCH₃ | H | H |
| II-A15 | 3 | S | —CH₂CH₂CH₂NH₂ | H | H |
| II-A16 | 3 | S | —CN | H | H |
| II-A17 | 3 | S | —H | H | H |
| II-A18 | 3 | S | —CN | H | H |
| II-A19 | 3 | S | —CH₂CO₂H | H | H |
| II-A20 | 3 | S | —CO₂CH₂CH₃ | H | H |
| II-A21 | 3 | S | —CH₂SO₂CH₃ | H | H |
| II-A22 | 3 | S | —CH₂NHSO₂CH₃ | H | H |
| II-A23 | 3 | S | —CH₂NHCOCH₃ | H | H |
| II-A24 | 3 | S | —CH₂CH₂OH | H | H |
| II-A25 | 3 | S | —COCH₂CH₃ | H | H |
| II-A26 | 3 | S | 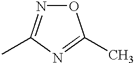 | H | H |
| II-A27 | 3 | S | 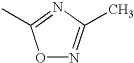 | H | H |
| II-A28 | 3 | S | 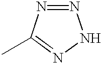 | H | H |
| II-A29 | 3 | S | 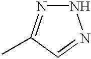 | H | H |
| II-A30 | 3 | S | 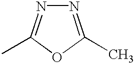 | H | H |
| II-A31 | 3 | S | 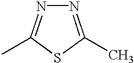 | H | H |
| II-A32 | 3 | S | 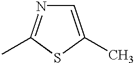 | H | H |
| II-A33 | 3 | S | 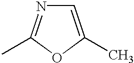 | H | H |
| II-A34 | 3 | S | 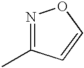 | H | H |
| II-A35 | 3 | S | 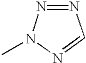 | H | H |
| II-A36 | 3 | S | 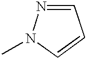 | H | H |
| II-A37 | 3 | S | 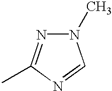 | H | H |
| II-A38 | 3 | S | 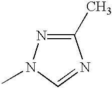 | H | H |
| II-A39 | 3 | S | 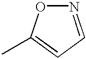 | H | H |
| II-A40 | 3 | S | 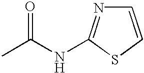 | H | H |
| II-A41 | 3 | S | 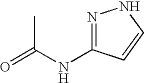 | H | H |
| II-A42 | 3 | S | 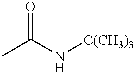 | H | H |
| II-A43 | 3 | S | 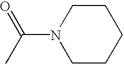 | H | H |
| II-A44 | 3 | S | 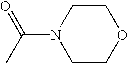 | H | H |
| II-A45 | 3 | S | 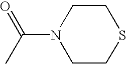 | H | H |
| II-A46 | 3 | S | 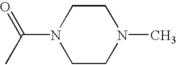 | H | H |
| II-A47 | 3 | S | 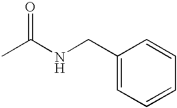 | H | H |
| II-A48 | 3 | S | —CH₂NHCONH₂ | H | H |
| II-A49 | 3 | S | 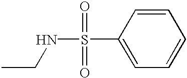 | H | H |
| II-A50 | 3 | S | —CN | H | 9-NH₂ |
| II-A51 | 3 | S | —CN | H | 9-NHCOCH₃ |
| II-A52 | 3 | S | —CN | H | 8-NH₂ |
| II-A53 | 3 | S | —CN | H | 8-NHCOCH₃ |
| II-A54 | 3 | S | —CN | H | 9-CH₃ |
| II-A55 | 3 | S | —CN | H | 8-OCH₃ |
| II-A56 | 3 | S | —CN | H | 8,9-Me₂ |
| II-A57 | 3 | S | —CN | H | 8-NHCO₂Me |
| II-A58 | 3 | S | —CN | H | 8-NMe₂ |
| II-A59 | 3 | S | —CN | CH₃ | H |
| II-A60 | 3 | S | —CN | CF₃ | H |
| II-A61 | 3 | S | —CN | Pr | H |
| II-A62 | 3 | S | —CN | Ph | H |
| II-A63 | 3 | S | —CN | CHMe₂ | H |
| II-A64 | 3 | S | —CN | NH₂ | H |
| II-A65 | 3 | S | —CN | CH₃ | H |
| II-A66 | 2 | S | —CN | CF₃ | H |
| II-A67 | 3 | S | —CN | CH₂Ph | H |
| II-A68 | 3 | O | —CN | H | H |
| II-A69 | 2 | O | —CN | H | H |
| II-A70 | 3 | O | —CN | CH₃ | H |
| II-A71 | 3 | O | —CN | cyclo-Pr | H |
| II-A72 | 3 | O | —CN | N(Me)CH₂Ph | H |
| II-A73 | 3 | O | —CO₂H | H | H |
| II-A74 | 3 | O | —CONH₂ | H | H |
| II-A75 | 3 | O | —H | H | H |
| II-A76 | 4 | O | —CN | H | H |
| II-A77 | 3 | S | —NH₂ | H | H |
| II-A78 | 3 | S | —NHR | H | H |
| II-A79 | 3 | S | —NHAc | H | H |
| II-A80 | 3 | S | —NHSO₂R | H | H |
| II-A81 | 3 | S | —NHCO₂R | H | H |
| II-A82 | 3 | S | —CONH₂ | H | H |

Another aspect of this embodiment relates to compounds of formula II wherein R¹ and R² are each acyclic substituents, said compounds referred to herein as compounds of formula II-B:

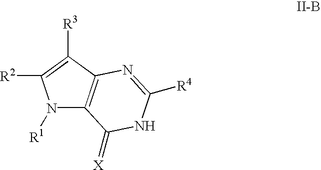

or a pharmaceutically acceptable derivative thereof, wherein R¹, R², R³, R⁴, and X are as defined above for formula I.

Preferred R¹, R², R³, and R⁴ groups for formula II-B are those described above for compounds of formula I.

Representative examples of compounds of formula II-B are shown below in Table 2.

TABLE 2
Examples of Compounds II-B

| No. | X | R¹ | R² | R³ | R⁴ |
|---|---|---|---|---|---|
| II-B1 | O | Et | Et | CN | H |
| II-B2 | S | Et | Et | CN | H |
| II-B3 | S | H | Et | CN | H |
| II-B4 | S | Ph | Et | CN | H |
| II-B5 | S | CH₂CH₂ (morpholin-4- | Et | CN | H |
| | | yl) | | | |
| II-B6 | S | isobutyl | isobutyl | CN | H |
| II-B7 | S | isobutyl | CF₃ | CN | H |
| II-B8 | S | CH₂Ph | CF₃ | CN | H |
| II-B9 | S | CH₂CH₂ (morpholin-4- | CF₃ | CN | H |
| | | yl) | | | |
| II-B10 | O | Ph | Me | CN | H |
| II-B11 | S | Ph | Me | CN | H |
| II-B12 | O | Ph | H | CN | H |
| II-B13 | S | Ph | H | CN | H |
| II-B14 | O | Et | Et | CN | H |
| II-B15 | O | H | Et | CN | H |
| II-B16 | S | CH₂CH₂Ph | Et | CN | H |
| II-B17 | O | Ph | Ph | CN | H |
| II-B18 | S | Ph | Ph | CN | H |
| II-B19 | S | COCH₃ | Et | CN | H |
| II-B20 | S | CONH₂ | Et | CN | H |
| II-B21 | S | CH₂CONH₂ | Et | CN | H |
| II-B22 | S | SO₂CH₃ | Et | CN | H |
| II-B23 | S | CH₂SO₂NH₂ | Et | CN | H |
| II-B24 | S | CO₂Et | Et | CN | H |
| II-B25 | S | cyclopropyl | Et | CN | H |
| II-B26 | S | Et | Ph | CN | H |
| II-B27 | O | Et | CH₂CH₂NH₂ | CN | H |
| II-B28 | S | isopropyl | isopropyl | CN | H |
| II-B29 | O | isobutyl | isobutyl | CN | H |
| II-B30 | O | Et | CH₂CH₂NHCbz | CN | H |
| II-B31 | S | Et | CH₂CH₂NHCbz | CN | H |
| II-B32 | O | Et | Ph | CN | H |

Another embodiment of this invention relates to compounds of formula I wherein R¹ and R² are taken together to form a dihydropyrido ring represented by formula II-C below:

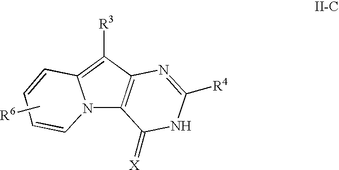

or a pharmaceutically acceptable derivative thereof, wherein R³, R⁴, R⁶, and X are as defined above for formula I.

Preferred R³, R⁴, and R⁶ groups for formula II-C are those described above for compounds of formula I.

Another embodiment of the present invention relates to compounds of formula II-D:

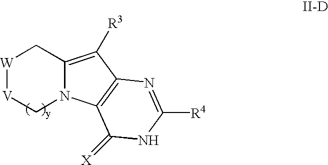

or a pharmaceutically acceptable derivative thereof, wherein X, R³, and R⁴ are as described above, y is 1-3, and W—V is selected from CH₂—NH, CH₂—O, CH₂—S, NH—CH₂, O—CH₂, S—CH₂, N═CH, or CH═N. Preferred substituents on any carbon on the ring bearing W—V is are selected from C₁₋₄ aliphatic, ═O, —OR, —CN, —CO₂R, —COR, —SO₂R, —C(═O)N(R)₂. Preferred substituents on any nitrogen of suitable valence on the ring bearing W—V are selected from C₁₋₄ aliphatic, CO(C₁₋₄ aliphatic), CO₂(C₁₋₄ aliphatic), or SO₂(C₁₋₄ aliphatic).

Preferred R³ and R⁴ groups of formula II-D are those described above for compounds of formula I.

Specific examples of compounds of formula II-D are shown in Table 3 below.

TABLE 3
Examples of Compounds II-D

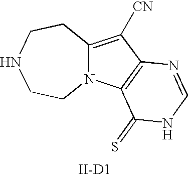

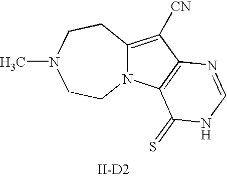

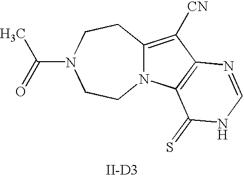

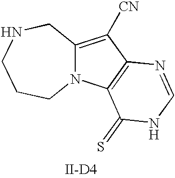

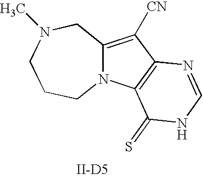

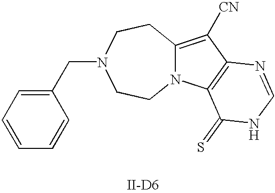

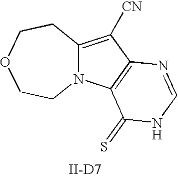

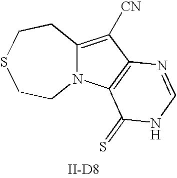

Another embodiment of this invention relates to compounds of formula I where Y is —S—, represented by compounds of formula III:

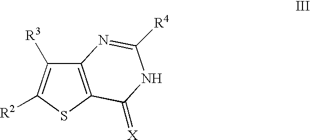

or a pharmaceutically acceptable derivative thereof, wherein R², R³, R⁴, and X are as defined above for formula I.

Preferred R², R³, and R⁴ groups for formula III are those described above for compounds of formula I.

Preferred compounds of formula III have one or more, and preferably all, of the features selected from the group consisting of:

(a) R² is selected from R, N(R)₂, OR, SR, C(O)R, CO₂R, C(O)N(R)₂, NRN(R)₂, NRC(O)R, SO₂R, or an optionally substituted 5-7 membered saturated, partially unsaturated, or fully unsaturated ring having 0-2 heteroatoms independently selected from nitrogen, oxygen, or sulfur;

(b) R³ is selected from R, CN, or Q₍ₙ₎R⁵, wherein n is zero or one, Q is selected from a C₁₋₄ alkylidene chain wherein one methylene unit of Q is optionally replaced by O, S, NR, C(O), CO₂, CONR, NRC(O), NRC(O)NR, SO₂, or NRSO₂, and R⁵ is selected from R or an optionally substituted 5-7 membered saturated, partially unsaturated, or fully unsaturated ring having 0-4 heteroatoms independently selected from nitrogen, oxygen, or sulfur; and

(c) R⁴ is selected from R, N(R)₂, or an optionally substituted 5-6 membered saturated, partially unsaturated, or fully unsaturated ring having 0-2 heteroatoms independently selected from nitrogen, oxygen, or sulfur.

Specific examples of compounds of formula III are shown in Table 4 below.

TABLE 4
Examples of compounds of formula III

| No. | X | R² | R³ | R⁴ |
|---|---|---|---|---|
| III-1 | S | H | CN | H |
| III-2 | S | NH₂ | CN | H |
| III-3 | S | NHCOCH₃ | CN | H |
| III-4 | O | SCH₃ | CN | H |
| III-5 | S | SCH₃ | CN | H |
| III-6 | S | SO₂CH₃ | CN | H |
| III-7 | S | NHCH₃ | CN | H |
| III-8 | S | SCH₂CH₃ | CN | H |
| III-9 | S | CH₂Ph | CN | H |
| III-10 | S | OCH(CH₃)₂ | CN | H |
| III-11 | S | CH₂CH₃ | CN | H |
| III-12 | S | 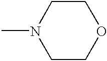 | CN | H |
| III-13 | S | 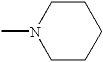 | CN | H |
| III-14 | S | 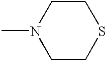 | CN | H |
| III-15 | S | 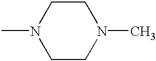 | CN | H |
| III-16 | S | 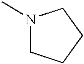 | CN | H |
| III-17 | S | 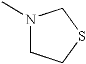 | CN | H |
| III-18 | S | 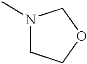 | CN | H |
| III-19 | S | 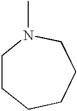 | CN | H |
| III-20 | S | N(Me)₂ | CN | H |
| III-21 | O | NHCH(CH₃)₂ | CN | H |
| III-22 | O | NHCH₂CH₂CH₃ | CN | H |
| III-23 | O | NHCH₂CH(CH₃)₂ | CN | H |
| III-24 | O | 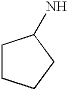 | CN | H |
| III-25 | O | 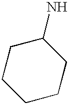 | CN | H |
| III-26 | O | NHCH₂Ph | CN | H |
| III-27 | S | NHSO₂R | CN | H |
| III-28 | O | NH₂ | CN | H |
| III-30 | O | NHCH(CH₃)₂ | C(═NH)NHCH(CH₃)₂ | H |
| III-31 | O | NHCH₂CH(CH₃)₂ | C(═NH)NHCH(CH₃)₂ | H |
| III-32 | O | NHNH₂ | CN | H |
| III-33 | O | 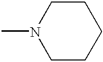 | CN | H |
| III-34 | O | 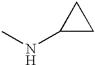 | CN | H |
| III-35 | O | 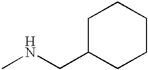 | CN | H |
| III-36 | O | NHCH₂CH₂CH(CH₃)₂ | CN | H |
| III-37 | O | 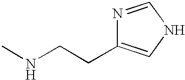 | CN | H |
| III-38 | O | CH₂CH₃ | CN | H |
| III-39 | O | N(CH₃)CH₂CH₂CH₃ | CN | H |

| | |
|---|---|
| III-40 | 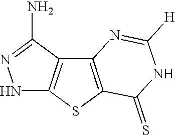 |

Compound III-40 is an example of a compound where R² and R³ are taken together to form an optionally substituted fused ring.

According to yet another embodiment, the present invention relates to a compound of formula IV:

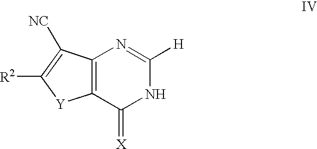

or a pharmaceutically acceptable derivative thereof, wherein:

X is oxygen or sulfur;

Y is —S— or —NR¹—;

R¹ is selected from R, CO₂R, C(O)R, CON(R)₂, SO₂R, SO₂N(R)₂, or an optionally substituted 5-7 membered monocyclic or 8-10 membered bicyclic saturated, partially unsaturated, or fully unsaturated ring having 0-3 heteroatoms independently selected from nitrogen, oxygen, or sulfur;

each R is independently selected from hydrogen or an optionally substituted C₁₋₆ aliphatic group;

R² is selected from R, N(R)₂, OR, SR, C(O)R, CO₂R, C(O)N(R)₂, NRN(R)₂, NRCOR, NRCO₂(C₁₋₆ aliphatic), NRSO₂(C₁₋₆ aliphatic), S(O)(C₁₋₆ aliphatic), SO₂R, SO₂N(R)₂, or an optionally substituted 5-7 membered monocyclic or 8-10 membered bicyclic saturated, partially unsaturated, or fully unsaturated ring system having 0-3 heteroatoms independently selected from nitrogen, oxygen, or sulfur, or:

when Y is —NR¹—, R¹ and R² are taken together to form a saturated, partially unsaturated, or fully unsaturated 4-9 membered mono- or bicyclic ring having 1-2 heteroatoms, in addition to the —NR¹— nitrogen, independently selected from nitrogen, oxygen, or sulfur, wherein said ring formed by R¹ and R² is optionally substituted with 1-2 R⁶; or

R⁵ is selected from R or an optionally substituted 5-14 membered mono-, bi-, or tricyclic aromatic, partially unsaturated, or saturated ring having 0-4 heteroatoms independently selected from nitrogen, oxygen, or sulfur; and

each R⁶ is independently selected from R, oxo, halogen, CN, C(O)R, CO₂R, SO₂R, OR, SR, N(R)₂, NRC(O)R, C(O)N(R)₂, NRCO₂R, OC(O)N(R)₂, NRSO₂R, or SO₂NR;

provided that if R¹ and R² taken together form a fused 5-7 membered ring, the fused ring contains more than one heteroatom.

Preferred R¹ and R² groups of formula IV are those described above for compounds of formula I.

The compounds of this invention may be prepared from known starting materials, by following known methods for analogous compounds, and by reference to the synthetic examples described below. References that are useful for making the present compounds include the following: Kadushkin, A. V. et al., Pharm. Chem. J., (1994) 28 (11), 792-798; Kadushkin, A. V. et al., Pharm. Chem. J., (1990) 24 (12), 875-881; Granik, V. G. et al., Chemistry of Heterocyclic Compounds (1982) 18(4), 321; Kadushkin, A. V. et al., Chem. Heterocycl. Compd. (English Translation), (1991) 27(3), 283-287; Stezhko, T. V. et al., Pharm. Chem. J. (Eng. Translation), (1985), 18(3), 154-161; Kadushkin, A. V. et al., Chem. Heterocycl. Compd. (English Translation), (1988), 23(12), 1297-1301; Kadushkin, A. V. et al., Pharm. Chem. J., (1987), 21(5), 317-322.

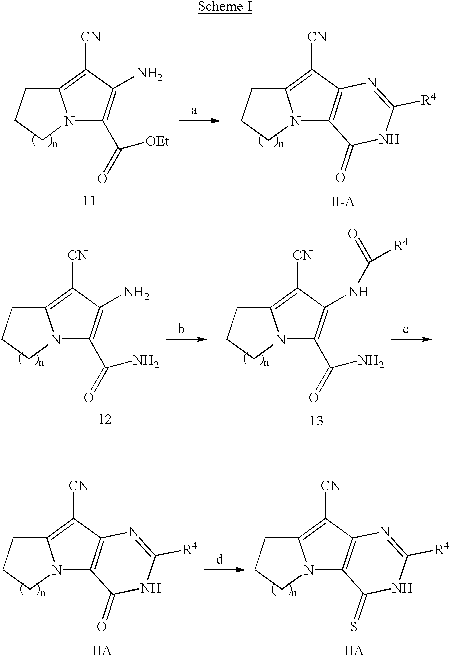

Reagents and conditions: (a) R⁴CN, acid catalyst; (b) R⁴COCl; (c) NaOEt, reflux; (d) i) POCl₃, Et₃N.HCl, 100° C.; ii) thiourea, toluene, 100° C.

Scheme I above shows alternative routes for preparing certain compounds of the present invention wherein R⁴ is an aliphatic group, an aryl or aralkyl group. For preparing compounds where R⁴ is NH₂, compound 11 is treated with cyanamide. The unsubstituted R⁴ amino group may be derivatized to provide further compounds of this invention. For example, treatment of II-A (X═O) where R⁴ is an unsubstituted amino group with R—CHO followed by treatment with NaBH₄ or R—COCl provides II-A where R⁴ is NH—R or NH—COR, respectively.

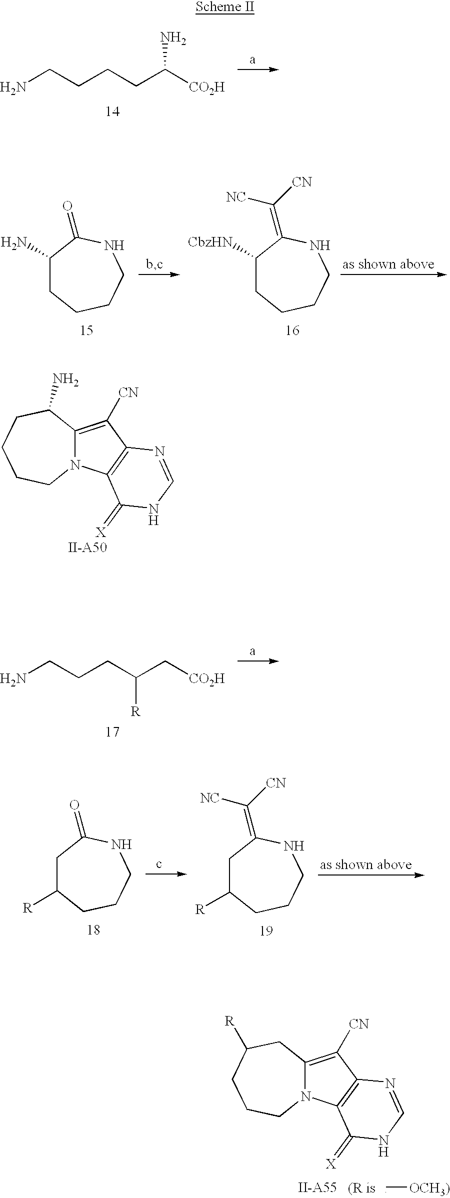

Reagents and conditions: (a) [(CH₃)₃Si]₂NH, catalytic (CH₃)₃SiCl, xylenes, reflux; (b) Cbz-Cl, (c) CH₂(CN)₂

Scheme II above shows a general route to compounds of formula II-A where the fused seven-membered ring formed by R¹ and R² is substituted. The route is illustrated starting with lysine (14) to provide the amino substituted II-A50. It would be apparent to one skilled in the art that lysine may be replaced by other (substituted)-6-aminocaproic acids to prepare other compounds of formula II-A where R¹ and R² form a seven membered ring that is substituted by various groups. The preparation of II-A52 shows a general route for introducing other substituents on the seven-membered ring.

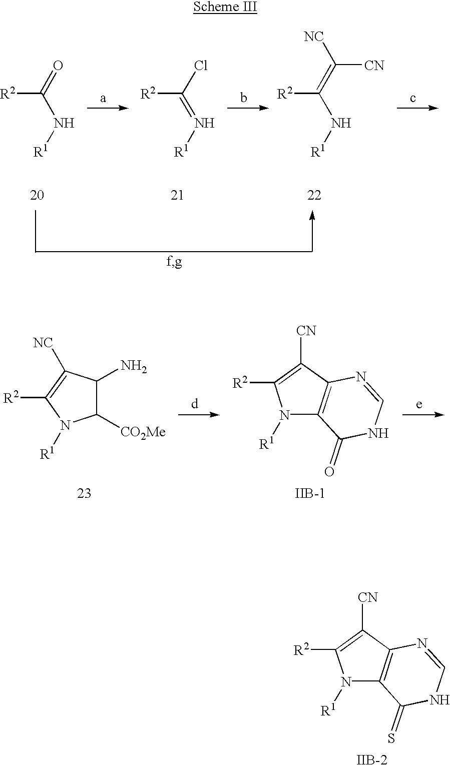

Reagents and conditions: (a) POCl₃, toluene, heat; (b) CH₂(CN)₂, Et₃N, CH₂Cl₂; (c) BrCH₂CO₂Me, K₂CO₃, DMF, heat; (d) i) DMF-DMA, DMF, 100° C.; ii) NH₃, MeOH, 100° C.; (e) i) POCl₃, Et₃N.HCl, 100° C.; ii) thiourea, toluene, 100° C. (f) (CH3)₃OBF₄, CH₂Cl₂ (g) CH₂(CN)₂, Et₃N, reflux.

Scheme III above shows a general approach to compounds of this invention where R¹ and R² are each independently selected from hydrogen or an optionally substituted aliphatic group. From intermediate 22 (prepared from Compound 20 using either steps a, b or f, g), the corresponding sequence of steps outlined above in either Scheme I or II from an analogous intermediate may be followed to provide II-B.

Procedures for carrying out these steps, or reactions analogous thereto are known. See Tamura, K. J. Org. Chem. (1993), 58, 32.

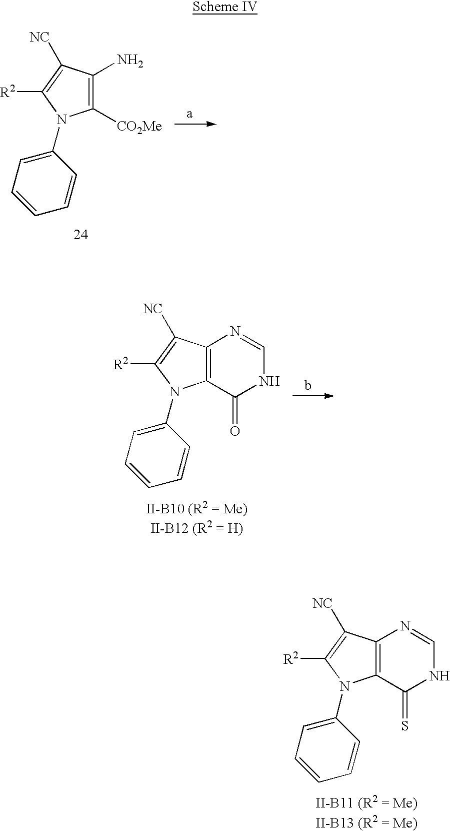

Reagents and conditions: (a) i) DMF-DMA, DMF, 100° C.; ii) NH₃, MeOH, 100° C.; (b) i) POCl₃, Et₃N.HCl, 100° C.; ii) thiourea, toluene, 100° C.

Scheme IV above shows a route to compounds of formula II-B where R¹ is aryl. Starting material 24 where R² is hydrogen or methyl is commercially available. Cyclization as described above provides II-B where X is oxygen, which are readily converted to compounds of formula II-B where X is sulfur.

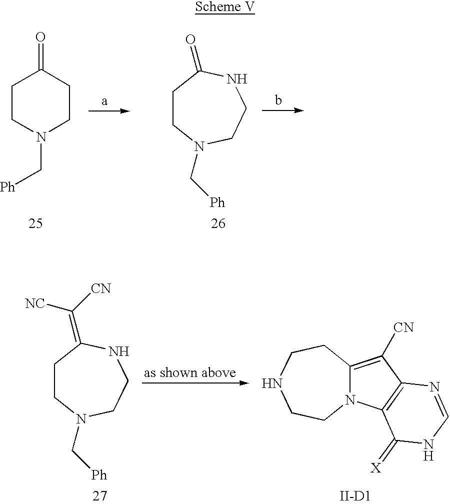

Reagents and conditions: (a) H₂N—OSO₃H, acetic acid, reflux; (b) CH₂(CN)₂

Scheme V above shows a route for preparing compounds of formula II-D where R¹ and R² taken together form a fused seven-membered ring having two heteroatoms. From intermediate 27, the sequence of steps outlined above in either Scheme I or II from an analogous intermediate may be followed to II-D. The NH in the seven-membered ring may be acylated or alkylated to provide further compounds of this invention. It also will be apparent to one skilled in the art that the NH may be replaced by oxygen or sulfur by an analogous route starting with either [1,4]oxazepan-3-one or [1,4]thiazepan-3-one, respectively.

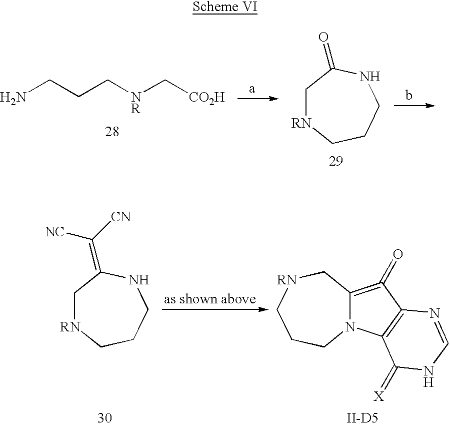

Reagents and conditions: (a) [(CH₃)₃Si]₂NH, catalytic (CH₃)₃SiCl, xylenes, reflux; (b) CH₂(CN)₂

Scheme VI above shows a route for preparing further compounds of formula II-D where R¹ and R² taken together form a fused seven-membered ring having two heteroatoms. From intermediate 30, the sequence of steps outlined above in either Scheme I or II from an analogous intermediate may be followed to II-D.

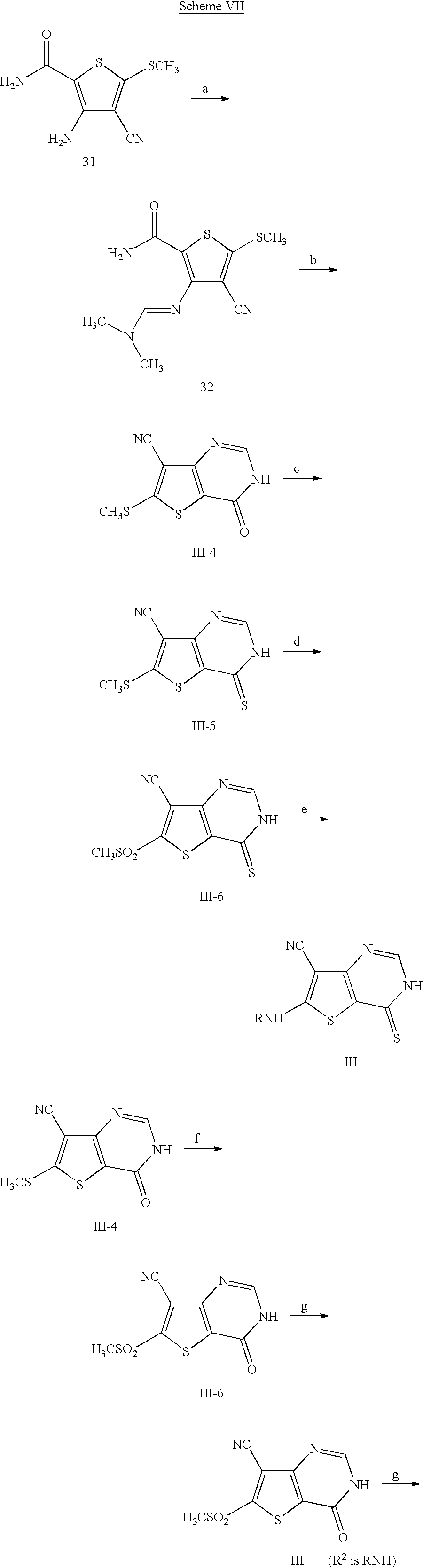

Reagents and conditions: (a) DMF-DMA, acetonitrile, 90° C.; (b) acetic acid, 90° C.; (c) Lawesson's Reagent; (d) Oxone®; (e) RNH₂, DMF; (f) mCPBA, CH₂Cl₂; (g) RNH₂, CH₃CN, 70° C.

Scheme VII above shows a route to compounds of this invention where Y is —S—. Procedures for these steps, or reactions analogous thereto, are known in the literature. See Briel, D., et al., J. Med. Chem. (1999) 42, 1849; Briel, D., et al., Pharmazie (1992) 47, 577-579 and Briel, D. Pharmazie (1998) 53, 227.

The details of the conditions used for producing these compounds are set forth in the Examples. One having ordinary skill in the art may synthesize other compounds of this invention following the teachings of the specification using reagents that are readily synthesized or commercially available.

The activity of a compound utilized in this invention as an inhibitor of GSK-3 may be assayed in vitro, in vivo or in a cell line. In vitro assays include assays that determine inhibition of either the phosphorylation activity or ATPase activity of activated GSK-3. Alternate in vitro assays quantitate the ability of the inhibitor to bind to GSK-3. Inhibitor binding may be measured by radiolabelling the inhibitor prior to binding, isolating the inhibitor/GSK-3 complex and determining the amount of radiolabel bound. Alternatively, inhibitor binding may be determined by running a competition experiment where new inhibitors are incubated with GSK-3 bound to known radioligands.

According to another embodiment, the invention provides a composition comprising a compound of this invention or a pharmaceutically acceptable salt thereof and a pharmaceutically acceptable carrier, adjuvant, or vehicle. The amount of compound in the compositions of this invention is such that is effective to detectably inhibit a protein kinase, particularly GSK-3 in a biological sample or in a patient. Preferably the composition of this invention is formulated for administration to a patient in need of such composition. Most preferably, the composition of this invention is formulated for oral administration to a patient.

The term “patient”, as used herein, means an animal, preferably a mammal, and most preferably a human.

The term “pharmaceutically acceptable carrier, adjuvant, or vehicle” refers to a non-toxic carrier, adjuvant, or vehicle that does not destroy the pharmacological activity of the compound with which it is formulated. Pharmaceutically acceptable carriers, adjuvants or vehicles that may be used in the compositions of this invention include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

The term “detectably inhibit”, as used herein means a measurable change in GSK-3 activity between a sample comprising said composition and a GSK-3 kinase and an equivalent sample comprising GSK-3 kinase in the absence of said composition.

A “pharmaceutically acceptable salt” means any non-toxic salt, ester, salt of an ester or other derivative of a compound of this invention that, upon administration to a recipient, is capable of providing, either directly or indirectly, a compound of this invention or an inhibitorily active metabolite or residue thereof.

Pharmaceutically acceptable salts of the compounds of this invention include those derived from pharmaceutically acceptable inorganic and organic acids and bases. Examples of suitable acid salts include acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, formate, fumarate, glucoheptanoate, glycerophosphate, glycolate, hemisulfate, heptanoate, hexanoate, hydrochloride, hydrobromide, hydroiodide, 2-hydroxyethanesulfonate, lactate, maleate, malonate, methanesulfonate, 2-naphthalenesulfonate, nicotinate, nitrate, oxalate, palmoate, pectinate, persulfate, 3-phenylpropionate, phosphate, picrate, pivalate, propionate, salicylate, succinate, sulfate, tartrate, thiocyanate, tosylate and undecanoate. Other acids, such as oxalic, while not in themselves pharmaceutically acceptable, may be employed in the preparation of salts useful as intermediates in obtaining the compounds of the invention and their pharmaceutically acceptable acid addition salts.

Salts derived from appropriate bases include alkali metal (e.g., sodium and potassium), alkaline earth metal (e.g., magnesium), ammonium and N⁺(C₁₋₄ alkyl)₄ salts. This invention also envisions the quaternization of any basic nitrogen-containing groups of the compounds disclosed herein. Water or oil-soluble or dispersible products may be obtained by such quaternization.

The compositions of the present invention may be administered orally, parenterally, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques. Preferably, the compositions are administered orally, intraperitoneally or intravenously. Sterile injectable forms of the compositions of this invention may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally-acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium.

For this purpose, any bland fixed oil may be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation.

The pharmaceutically acceptable compositions of this invention may be orally administered in any orally acceptable dosage form including, but not limited to, capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include lactose and dried cornstarch. When aqueous suspensions are required for oral use, the active ingredient is combined with emulsifying and suspending agents. If desired, certain sweetening, flavoring or coloring agents may also be added.

Alternatively, the pharmaceutically acceptable compositions of this invention may be administered in the form of suppositories for rectal administration. These can be prepared by mixing the agent with a suitable non-irritating excipient that is solid at room temperature but liquid at rectal temperature and therefore will melt in the rectum to release the drug. Such materials include cocoa butter, beeswax and polyethylene glycols.

The pharmaceutically acceptable compositions of this invention may also be administered topically, especially when the target of treatment includes areas or organs readily accessible by topical application, including diseases of the eye, the skin, or the lower intestinal tract. Suitable topical formulations are readily prepared for each of these areas or organs.

Topical application for the lower intestinal tract can be effected in a rectal suppository formulation (see above) or in a suitable enema formulation. Topically-transdermal patches may also be used.

For topical applications, the pharmaceutically acceptable compositions may be formulated in a suitable ointment containing the active component suspended or dissolved in one or more carriers. Carriers for topical administration of the compounds of this invention include, but are not limited to, mineral oil, liquid petrolatum, white petrolatum, propylene glycol, polyoxyethylene, polyoxypropylene compound, emulsifying wax and water. Alternatively, the pharmaceutically acceptable compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water.

For ophthalmic use, the pharmaceutically acceptable compositions may be formulated as micronized suspensions in isotonic, pH adjusted sterile saline, or, preferably, as solutions in isotonic, pH adjusted sterile saline, either with or without a preservative such as benzylalkonium chloride. Alternatively, for ophthalmic uses, the pharmaceutically acceptable compositions may be formulated in an ointment such as petrolatum.

The pharmaceutically acceptable compositions of this invention may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

Most preferably, the pharmaceutically acceptable compositions of this invention are formulated for oral administration.

The amount of the compounds of the present invention that may be combined with the carrier materials to produce a composition in a single dosage form will vary depending upon the host treated, the particular mode of administration. Preferably, the compositions should be formulated so that a dosage of between 0.01-100 mg/kg body weight/day of the inhibitor can be administered to a patient receiving these compositions.

It should also be understood that a specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease being treated. The amount of a compound of the present invention in the composition will also depend upon the particular compound in the composition.

Depending upon the particular condition, or disease, to be treated or prevented, additional therapeutic agents, which are normally administered to treat or prevent that condition, may also be present in the compositions of this invention. As used herein, additional therapeutic agents that are normally administered to treat or prevent a particular disease, or condition, are known as “appropriate for the disease, or condition, being treated”.

For example, chemotherapeutic agents or other anti-proliferative agents may be combined with the compounds of this invention to treat proliferative diseases and cancer. Examples of known chemotherapeutic agents include, but are not limited to, Gleevec™, adriamycin, dexamethasone, vincristine, cyclophosphamide, fluorouracil, topotecan, taxol, interferons, and platinum derivatives.

Other examples of agents the compounds of this invention may also be combined with include, without limitation, anti-inflammatory agents such as corticosteroids, TNF blockers, IL-1 RA, azathioprine, cyclophosphamide, and sulfasalazine; immunomodulatory and immunosuppressive agents such as cyclosporin, tacrolimus, rapamycin, mycophenolate mofetil, interferons, corticosteroids, cyclophophamide, azathioprine, and sulfasalazine; neurotrophic factors such as acetylcholinesterase inhibitors, MAO inhibitors, interferons, anti-convulsants, ion channel blockers, riluzole, and anti-Parkinsonian agents; agents for treating cardiovascular disease such as beta-blockers, ACE inhibitors, diuretics, nitrates, calcium channel blockers, and statins; agents for treating liver disease such as corticosteroids, cholestyramine, interferons, and anti-viral agents; agents for treating blood disorders such as corticosteroids, anti-leukemic agents, and growth factors; agents for treating diabetes such as insulin, insulin analogues, alpha glucosidase inhibitors, biguanides, and insulin sensitizers; and agents for treating immunodeficiency disorders such as gamma globulin.

The amount of additional therapeutic agent present in the compositions of this invention will be no more than the amount that would normally be administered in a composition comprising that therapeutic agent as the only active agent. Preferably the amount of additional therapeutic agent in the presently disclosed compositions will range from about 50% to 100% of the amount normally present in a composition comprising that agent as the only therapeutically active agent.

According to another embodiment, the invention relates to a method of inhibiting GSK-3 kinase activity in a biological sample comprising the step of contacting said biological sample with a compound of this invention, or composition comprising said compound.

The term “biological sample”, as used herein, includes, without limitation, cell cultures or extracts thereof; biopsied material obtained from a mammal or extracts thereof; and blood, saliva, urine, feces, semen, tears, or other body fluids or extracts thereof.

Inhibition of GSK-3 kinase activity in a biological sample is useful for a variety of purposes which are known to one of skill in the art. Examples of such purposes include, but are not limited to, blood transfusion, organ-transplantation, biological specimen storage, and biological assays.

According to another embodiment, the invention provides a method for treating or lessening the severity of a GSK-3-mediated disease or condition in a patient comprising the step of administering to said patient a composition according to the present invention.

The term “GSK3-mediated disease”, as used herein means any disease or other deleterious condition in which GSK3 is known to play a role. Accordingly, these compounds are useful for treating diseases or conditions that are known to be affected by the activity of GSK3 kinase. Such diseases or conditions include, but are not limited to, diabetes, neurodegenerative diseases, AIDS associated dementia, multiple sclerosis (MS), schizophrenia, cardiomycete hypertrophy, and baldness.

Neurodegenerative diseases which may be treated or prevented by the compounds of this invention include, but are not limited to, Alzheimer's disease, Parkinson's disease, amyotrophic lateral sclerosis (ALS), epilepsy, seizures, Huntington's disease, traumatic brain injury, ischemic and hemorrhaging stroke, or cerebral ischemias.

Another preferred embodiment relates to the method used to treat or prevent a GSK3-mediated disease selected from diabetes, Alzheimer's disease, Huntington's disease, Parkinson's disease, multiple sclerosis (MS), or amyotrophic lateral sclerosis (AML).

Certain compounds of the present invention are also inhibitors of ROCK kinase. In particular, compounds of formula III are inhibitors of ROCK kinase. Accordingly, another embodiment of the present invention relates to a method of inhibiting ROCK kinase in a biological sample comprising the step of contacting said biological sample with a compound of formula III, or composition comprising said compound.

According to another embodiment, the invention provides a method for treating or lessening the severity of a ROCK-mediated disease or condition in a patient comprising the step of administering to said patient a compound of formula III, or composition comprising said compound.

The term “ROCK-mediated disease”, as used herein means any disease or other deleterious condition in which ROCK is known to play a role. Accordingly, these compounds are useful for treating diseases or conditions that are known to be affected by the activity of ROCK kinase. Such diseases or conditions include, but are not limited to, hypertension, erectile dysfunction, angiogenesis, neuroregeneration, metastasis, glaucoma, inflammation, artheriosclerosis, immunosuppresion, restenosis, asthma, and cardiac hypertrophy.

In addition to the compounds of this invention, pharmaceutically acceptable derivatives the compounds of this invention may also be employed in compositions to treat or prevent the above-identified disorders.

In an alternate embodiment, the methods of this invention that utilize compositions that do not contain an additional therapeutic agent, comprise the additional step of separately administering to said patient an additional therapeutic agent. When these additional therapeutic agents are administered separately they may be administered to the patient prior to, sequentially with or following administration of the compositions of this invention.

The compounds of this invention or pharmaceutical compositions thereof may also be incorporated into compositions for coating an implantable medical device, such as prostheses, artificial valves, vascular grafts, stents and catheters. Vascular stents, for example, have been used to overcome restenosis (re-narrowing of the vessel wall after injury). However, patients using stents or other implantable devices risk clot formation or platelet activation. These unwanted effects may be prevented or mitigated by pre-coating the device with a pharmaceutically acceptable composition comprising a kinase inhibitor. Suitable coatings and the general preparation of coated implantable devices are described in U.S. Pat. Nos. 6,099,562; 5,886,026; and 5,304,121. The coatings are typically biocompatible polymeric materials such as a hydrogel polymer, polymethyldisiloxane, polycaprolactone, polyethylene glycol, polylactic acid, ethylene vinyl acetate, and mixtures thereof. The coatings may optionally be further covered by a suitable topcoat of fluorosilicone, polysaccarides, polyethylene glycol, phospholipids or combinations thereof to impart controlled release characteristics in the composition. Implantable devices coated with a compound of this invention are another embodiment of the present invention.

In order that the invention described herein may be more fully understood, the following examples are set forth. It should be understood that these examples are for illustrative purposes only and are not to be construed as limiting this invention in any manner.

### SYNTHETIC EXAMPLES

### Example 1

### 4-Thioxo-3,4,5,6,7,8-hexahydro-1,3,4b-triaza-fluorene-9-carbonitrile (II-A2)

A mixture of commercially available 4-chloro-5,6,7,8-tetrahydro-1,3,4b-triaza-fluorene-9-carbonitrile (0.05 g, 0.21 mmol)and thiourea (0.02 g, 0.27 mmol) in toluene (5 mL) was heated in a sealed tube at 110-115° C. for two hours. Additional thiourea (0.02 g, 0.27 mmol) was added and heating continued an additional 2 hours. The reaction was cooled and stirred with 2N sodium hydroxide (9 mL) for 10 minutes. Separation and acidification of the aqueous phase (6N hydrochloric acid) was followed by extraction with three portions of ethyl acetate. The organic phase was washed with brine, was dried (sodium sulfate) and was evaporated. Purification by flash chromatography (SiO₂) eluted with 2:98 methanol:dichloromethane provided the title compound (0.04 g, 78% yield) as a white solid. ¹HNMR (500 MHz, DMSO-d6) δ7.90 (s, 1H), 4.61 (m, 2H), 2.85 (m, 2H), 1.81 (m, 2H), 1.66 (m, 2H) ppm. MS (ES+): m/e=231.05 (M+H).

### Example 2

### 4-Thioxo-3,4,5,6,7,8,9,10-octahydro-1,3,4b-triaza-cycloocta[a]indene-11-carbonitrile (II-A4)

Step A. 2-Azacan-2-ylidene-malonitrile

A solution of azacan-2-one (0.50 g, 3.93 mmol) in dichloromethane (4 mL) was treated with trimethyloxonium tetrafluoroborate (0.70 g, 4.72 mmol) and stirred at room temperature under nitrogen for 5 hours. The solvent was evaporated and to the residue was added ethanol (20 mL), triethylamine (0.68 mL, 5.11 mmol) and malononitrile (0.28 mL, 4.32 mmol). The reaction was refluxed for 3 hours, cooled to room temperature, then diluted with ethyl acetate. This was washed with 10% potassium bisulfate and brine, dried (sodium sulfate) and evaporated. Purification by flash chromatography (SiO₂) eluted with 3:7 ethyl acetate:hexanes provided-the title compound (0.16 g, 23% yield) as a white-solid. ¹HNMR (500 MHz, DMSO-d6) δ8.73 (br s, 1H), 3.34 (m, 2H), 2.52 (m, 2H), 1.62 (m, 2H), 1.45 (m, 2H), 1.34 (m, 2H) ppm.

Step B. 2-Amino-1-cyano-5,6,7,8,9,10-hexahydro-pyrrole[1,2a]azocine-3-carboxylic acid methyl ester

This compound was prepared using the procedure described in Example 13, Step B, except starting with 2-azacan-2-ylidene-malonitrile (0.49 g, 2.77 mmol) to the title compound (0.32 g, 47% yield) as an off-white solid. ¹HNMR (500 MHz, CDCl3) δ4.81 (br s, 2H), 4.24 (m, 2H), 3.78 (s, 3H), 2.69 (m, 2H), 1.69 (m, 4H), 1.44 (m, 2H), 1.10 (m, 2H) ppm. MS (ES+): m/e=248.07 (M+H).

Step C. 4-Oxo-3,4,5,6,7,8,9,10-octahydro-1,3,4b-triaza-cycloocta[a]indene-11-carbonitrile

This compound was prepared using the procedure described in Example 9, except starting with 2-amino-1-cyano-5,6,7,8,9,10-hexahydro-pyrrole[1,2a]azocine-3-carboxylic acid methyl ester (0.31 g, 1.25 mmol) to afford the title compound (0.26 g, 86% yield) as a white solid. ¹HNMR (500 MHz, DMSO-d6) δ12.4 (br s, 1H), 7.99 (s, 1H), 4.57 (m, 2H), 3.01 (m, 2H), 1.78 (m, 4H), 1.49 (m, 2H), 1.14 (m, 2H) ppm. MS (ES+): m/e=243.08 (M+H).

Step D. 4-Thioxo-3,4,5,6,7,8,9,10-octahydro-1,3,4b-triaza-cycloocta[a]indene-11-carbonitrile (II-A4)

This compound was prepared using the procedure described in Example 11, except starting with 4-oxo-3,4,5,6,7,8,9,10-octahydro-1,3,4b-triaza--cycloocta[a]indene-11-carbonitrile (0.23 g, 0.95 mmol) to provide the title compound (0.05 g, 766 yield) as a yellow solid. ¹HNMR (500 MHz, DMSO-d6) δ13.6 (br s, 1H), 8.09 (s, 1H), 4,98 (br s, 2H), 3.00 (br s, 2H), 1.80 (br, 2H), 1.71 (br s, 2H), 1.46 (br s, 2H), 1.03 (br s, 2H) ppm. MS (ES+): m/e=259.06 (M+H).

### Example 3

### 6,7,8,9-Tetrahydro-3H,5H-1,3,4b-triaza-benzo[a]azulene-4-thione (II-A17)

4-Thioxo-4,5,6,7,8,9-hexahydro-3H-1,3,4b-triaza-benzo[a]azulene-10-carbonitrile (100 mg, 41 mmol) was suspended in a solution of polyphosphoric acid (obtained from 1.4 g phosphorus pentoxide and 6 mL of concentrated phosphoric acid) and heated to 200° C. for 18 hours. The reaction was cooled to room temperature and poured onto 50 mL crushed ice. The resulting slurry was basified to pH8 using 6N NaOH, and this aqueous layer was extracted with dichloromethane (3×30 mL). The organic layer was dried over Na₂SO₄, evaporated, and the resulting residue was purified by flash chromatography on silica gel (90/10 dichloromethane/methanol) to yield 21 mg (24% yield) of the desired product. ¹H NMR (500 MHz, DMSO-d6) δ13.12 (s, 1H), 7.99 (s, 1H), 6.35 (s, 1H), 5.41 (s, 2H), 3.41 (s, 2H) 2.97 (s, 2H), 1.85 (s, 2H), 1.65 (s, 2H). MS (M+H) 220.02.

### Example 4

### N-Methyl-4-thioxo-4,5,6,7,8,9-hexahydro-3H-1,3,4b-triaza-benzo[a]azulene-10-carbonitrile (II-A59)

Step A. N-Methyl-4-oxo-4,5,6,7,8,9-hexahydro-3H-1,3,4b-triaza-benzo[a]azulene-10-carbonitrile (II-A70)

A solution 2-amino-1-cyano-6,7,8,9-tetrahydro-5H-pyrrolo[1,2-a]azepine-3-carboxylic acid, prepared according to literature methods (Kadushkin, A. V. et al., Pharm. Chem. J., (1990) 24 (12), 875-881)(760 mg, 3.07 mmol),and N,N-dimethylacetamide dimethylacetal (900 μL, in 4.95, mmol) in dimethylformamide (10 mL) was heated at 100° C. for 5.5 hours, then evaporated. The intermediate was dissolved in MeOH (5 ml) and treated with 7N ammonia in methanol (10 mL), and heated in a sealed tube at 110° C. for 3days. The reaction was cooled, and the precipitate filtered to give the title compound as a brown solid (647 mg, 34% yield). ¹HNMR (500 MHz, CD₃OD) δ4.67-4.88 (m, 2H), 2.90-3.11 (m, 2H), 2.45 (s, 3H), 1.89-2.03 (m, 2H), 1.71-1.88 (m, 4H) ppm. LC−MS (ES+): m/e=243.08 (M+H). Analytical HPLC (cyano column); 6.71 min.

Step B. N-Methyl-4-thioxo-4,5,6,7,8,9-hexahydro-3H-1,3,4b-triaza-benzo[a]azulene-10-carbonitrile (II-A59)

A mixture of N-methyl-4-oxo-4,5,6,7,8,9-hexahydro-3H-1,3,4b-triaza-benzo[a]azulene-10-carbonitrile (0.079 g, 0.33 mmol) and triethylamine hydrochloride (0.05 g, 0.36 mmol) in phosphorous oxychloride (2.5 mL) in a sealed tube was heated at 100° C. for 1 hour. After cooling, the solvent was evaporated, the residue was treated with water, adjusted to pH 9 with potassium carbonate and with ethyl acetate (3×5 ml). The organic phase was dried over sodium sulfate and was evaporated to provide the intermediate (0.061 g) as a white solid. The intermediate (0.030 g, 0.115 mmol) was dissolved in toluene (2.5mL) and was treated with thiourea (0.013 g, 0.17 mmol), then heated at 100° C. in a sealed tube for 1.5 hours. The reaction was cooled and stirred with 10% (w/v) sodium hydroxide (5 mL) for 15 minutes. Separation and acidification (pH1) of the aqueous phase (6N hydrochloric acid) was followed by extraction with three portions of ethyl acetate. The organic phase was dried over sodium sulfate and was evaporated. Flash chromatography on silica, eluted first with 2% methanol in dichloromethane, provided the title compound as a white solid (0.01 g, 34% yield). ¹HNMR (500 MHz, CD₃OD) δ5.40-5.55 (m, 2H), 2.96-3.18 (m, 2H), 2.48 (s, 3H), 1.84-2.04 (m, 2H), 1.64-1.85 (m, 4H) ppm. MS (ES+): m/e=259.05 (M+H). LC−MS (cyano column) 6.29 min.

### Example 5

### 2-Cyclopropyl-4-oxo-4,5,6,7,8,9-hexahydro-3H-1,3,4b-triaza-benzo[a]azulene-10-carbonitrile (IIA-71)

A solution 2-amino-1-cyano-6,7,8,9-tetrahydro-5H-pyrrolo[1,2-a]azepine-3-carboxylic acid, prepared according to literature methods (Kadushkin, A. V. et al., Pharm. Chem. J., (1990) 24 (12), 875-881)(0.221 g, 0.89 mmol) and cyclopropyl cyanide (400 μL, 5.43 mmol) in 4N HCl in dioxane (4 mL) was heated at 110° C. for 3 hours. The precipitate that formed was filtered (55 mg). The intermediate was dissolved in 7N HCl in MeOH (4 ml) and heated in a sealed tube at 110° C. for 18 hours. The reaction was cooled, and the solvent was evaporated. The crude product was purified by flash column chromatography (SiO₂), eluting with 1-5% MeOH in dichloromethane to give the title compound as a white solid (10 mg, 4% yield). ¹HNMR (500 MHz, CD₃OD) δ4.76-4.85 (m, 2H), 4.08-4.19 (m, 2H), 3.09-3.20 (m, 2H), 2.99-3.09 (m, 2H), 2.22-2.37 (m, 2H), 1.86-1.99 (m,2H), 1.67-1.86 (m, 4H) ppm. LC−MS (ES+): m/e=269.04 (M+H). Analytical HPLC (cyano column); 8.26 min. IR (cm⁻¹) 2217 (CN stretch).

### Example 6

### N-(10-Cyano-4-oxo-4,5,6,7,8,9-hexahydro-3H-1,3,4b-triaza-benzo[a]azulen-2-yl)-N-methylbenzamide (II-A72)

A solution 2-amino-1-cyano-6,7,8,9-tetrahydro-5H-pyrrolo[1,2-a]azepine-3-carboxylic acid (0.24 g, 0.97 mmol) and benzoyl isothiocyanate (160 μL, 1.18 mmol) in CH₂Cl₂ (10 ml) was stirred at room temperature for 3 hours. The solvent was evaporated and the resulting solid was triturated with hexanes (3×5 ml) to give a brown solid. This intermediate was dissolved in CH₂Cl₂ (2mL) and was treated with DBU (100 μL, 0.67 mmol) and iodomethane (40 μL 0.64 mmol) and the solution was stirred at room temperature for 18 hours. The crude product was purified by flash column chromatography (SiO₂), eluting with 1% MeOH in dichloromethane to give a yellow oil (44 mg). The intermediate (44 mg, 0.10 mmol) was dissolved in 7N NH₃ in MeOH (3 mL) and heated at 110° C. for 1 h in a sealed tube. Cooled to room temperature affording a white precipitate. The precipitate was filtered to give the title compound as white solid (6 mg, 17%). ¹HNMR (500 MHz, CD₃OD) δ13.87 (s, 1H), 8.12-8.44 (d, J=7.2 Hz, 2H), 7.32-7.62 (m, 3H), 4.56-4.94 (broad s, 2H), 4.06 (s, 3H), 2.87 (m,2H), 1.68-2.04 (m, 6H) ppm. LC−MS (ES+): m/e=362.17 (M+H).

### Example 7

### 4-Oxo-4,5,6,7,8,9-hexahydro-3H-1,3,4b-triaza-benzo[a]azulene-10-carboxylic acid amide (II-A74)

4-Oxo-4,5,6,7,8,9-hexahydro-3H-1,3,4b-triaza-benzo[a]azulene-10-carbonitrile (110 mg, 48 mmol) was suspended in a solution of 6N hydrochloric acid (25 mL) and glacial acetic acid (15 mL). The solution was heated to 50° C. for 4 hours, after which 5 drops of concentrated sulfuric acid were added, and the solution was stirred for an additional 30 min. The solvent was evaporated, and the residue was treated with cold water, which caused the product to precipitate. The precipitate was filtered and dried at 50° C. for 24 hours, affording 76 mg (65% yield) of the title compound. ¹H NMR (500 MHz, DMSO-d6): 12.45 (s, 1H), 8.17 (s, 1H), 7.91 (s, 1H), 7.20 (s, 1H), 4.70 (s, 2H), 3.43 (s, 2H), 1.77 (s, 2H), 1.59 (S, 2H), 1.51 (s, 2H). MS (M+H) 247.12.

### Example 8

### 6,7,8,9-Tetrahydro-3H,5H-1,3,4b-triaza-benzo[a]azulen-4-one (II-A75)

4-Oxo-4,5,6,7,8,9-hexahydro-3H-1,3,4b-triaza-benzo[a]azulene-10-carbonitrile (50 mg, 22 mmol) was suspended in a solution of polyphosphoric acid (obtained from 700 mg of phosphorus pentoxide and 3 mL of concentrated phosphoric acid) and heated to 200° C. while stirring for 5 hours. The reaction was cooled to room temperature and poured into 50 mL of crushed ice. The resulting slurry was basified to pH 8 using 6N NaOH. The aqueous layer was extracted with 3×20 mL of dichloromethane, and this organic layer was washed with brine, dried over Na₂SO₄, and evaporated. The residue was purified by flash chromatography on silica gel (90/10 dichloromethane/methanol) to yield 30 mg (68% yield) of the desired product. ¹H NMR (500 MHz, DMSO-d6): 11.81 (s, 1H), 7.73 (s, 1H), 6.13 (s, 1H), 4.71 (s, 2H), 3.33 (s, 1H), 2.82 (s, 2H), 1.80 (s, 2H), 1.67 (s, 3H). MS (M+H) 204.04.

### Example 9

### 6-Methyl-4-oxo-5-phenyl-4,5-dihydro-3H-pyrrolo[3,2-d]pyrimidine-7-carbonitrile (II-B10)

A solution of 3-amino-4-cyano-5-methyl-1-phenyl-1H-pyrrole-2-carboxylic acid methyl ester (0.10 g, 0.38 mmol) and dimethylformamide dimethylacetal (0.10 mL, 0.75 mmol) in dimethylformamide (2 mL) was heated at 100-105° C. for 1.5 h, then evaporated. The intermediate was dissolved in methanol (2 mL), was treated with 7N ammonia in methanol (5 mL), was sealed in a tube and was heated at 100-105° C. for 3 hours. The reaction was cooled, was evaporated and was purified by flash chromatography (SiO₂) eluted with 1:99 methanol:dichloromethane to provide the title compound (0.08 g, 82% yield) as a white solid. ¹HNMR (500 MHz, DMSO-d6) δ12.4 (br s, 1H), 8.08 (s, 1H), 7.60 (m, 3H), 7.54 (m, 2H), 2.35 (s, 3H) ppm. MS (ES+): m/e=251.10 (M+H).

### Example 10

### 4-Oxo-5-phenyl-4,5-dihydro-3H-pyrrolo[3,2-d]pyrimidine-7-carbonitrile (II-B12)

Was prepared in a manner analogous to that described in Example 9. ¹HNMR (500 MHz, DMSO-d6) δ12.4 (br s, 1H), 8.46 (s, 1H), 8.02 (s, 1H), 7.50 (m, 5H) ppm. MS (ES+): m/e=236.98 (M+H).

### Example 11

### 6-Methyl-5-phenyl-4-thioxo-4,5-dihydro-3H-pyrrolo[3,2-d]pyrimidine-7-carbonitrile 3 (II-B11)

A mixture of 6-methyl-4-oxo-5-phenyl-4,5-dihydro-3H-pyrrolo[3,2-d]pyrimidine-7-carbonitrile (Compound II-B10)(0.06 g, 0.23 mmol) and triethylamine hydrochloride (0.03 g, 0.24 mmol) in phosphorous oxychloride (2 mL) in a sealed tube was heated at 100-105° C. for 1 hours. After cooling, the solvent was evaporated, the residue was treated with water, adjusted to pH 9 with potassium carbonate and was extracted with ethyl acetate (3×). The organic phase was dried over sodium sulfate and was evaporated to provide the intermediate (0.06 g) as a white solid. The intermediate was dissolved in toluene (3 mL) and was treated with thiourea (0.02 g, 0.29 mmol), then heated at 100-105° C. in a sealed tube for 4 hours. The reaction was cooled and stirred with 2N sodium hydroxide (9 mL) for 10 minutes. Separation and acidification of the aqueous phase (6N hydrochloric acid) was followed by extraction with three portions of ethyl acetate. The organic phase was washed with brine, was dried (sodium sulfate) and was evaporated. Purification by two flash chromatographies (SiO₂) eluted first with 0.75-1.5% methanol in dichloromethane, then with 1:1 ethyl acetate:hexanes to provide the title compound (0.03 g, 49% yield) as a pale yellow solid. ¹HNMR (500 MHz, DMSO-d6) δ13.7 (br s, 1H), 8.28 (s, 1H), 7.62 (m, 3H), 7.52 (m, 2H), 2.37 (s, 3H) ppm. MS (ES+): m/e=267.01 (M+H).

### Example 12

### 5-phenyl-4-thioxo-4,5-dihydro-3H-pyrrolo[3,2-d]pyrimidine-7-carbonitrile (II-B13)

Was prepared in an analogous manner: ¹HNMR (500 MHz, DMSO-d6) δ14.0 (br s, 1H), 8.85 (s, 1H), 8.43 (s, 1H), 7.68 (m, 5H) ppm. MS (ES+): m/e=252.99 (M+H).

### Example 13

### 5,6-Diethyl-4-thioxo-4,5-dihydro-3H-pyrrolo[3,2-d]pyrimidine-7-carbonitrile (II-B2)

Step A. 2-(1-Ethylamino-propylidene)malononitrile

A solution of N-ethylpropionamide 9 (1.0 g, 9.9 mmol) in toluene (5 mL) was treated with a solution of phosphorous oxychloride (0.92 mL, 9.9 mmol) in toluene (5 mL) over 2 minutes and stirred at room temperature under nitrogen for 2 hours. Over 10 minutes was added a solution of malonitrile (0.63 mL, 9.9 mmol) and triethylamine (1.65 mL, 11.9 mmol) in dichloromethane (15 mL). The resulting solution was stirred at room temperature for 3 days. The reaction was washed with saturated sodium bicarbonate and with 10% potassium bisulfate, was dried (sodium sulfate) and was evaporated. Purification by flash chromatography (SiO₂) eluted with 35:65 ethyl acetate:hexanes provided the title compound (0.38 g, 26% yield) as a colorless semi-solid. ¹HNMR (500 MHz, CDCl₃) δ6.20 (br s, 1H), 3.35 (dq, J=7.1, 7.0 Hz, 2H), 2.51 (q, J=7.6 Hz, 2H), 1.24 (t, J=7.2 Hz, 3H), 1.20 (t, J=7.7 Hz, 3H) ppm. MS (ES+): m/e=150.02 (M+H).

Step B. 3-Amino-4-cyano-1,5-diethyl-1H-pyrrole-2-carboxylic acid methyl ester

To a suspension of the above prepared 2-(1-ethylamino-propylidene)malonitrile (0.38 g, 2.51 mmol) and potassium carbonate (0.38 g, 2.76 mmol) in dimethylformamide (5 mL) was added methyl bromoacetate (0.25 mL, 2.64 mmol). The reaction was stirred at 100-105° C. under nitrogen for 4 hours, and was cooled. The reaction was diluted with ethyl acetate, was washed with four portions of water and one of brine, was dried (sodium sulfate) and was evaporated. Purification by flash chromatography (SiO₂) eluted with 2:8 ethyl acetate:hexanes provided the title compound (0.37 g, 67% yield) as a white solid. ¹H NMR (500 MHz, CDCl3) δ4.89 (br s, 2H), 4.25 (q, J=7.1 Hz, 2H), 3.88 (s, 3H), 2.72 (q, J=7.6 Hz, 2H), 1.31 (m, 6H) ppm. MS (ES+): m/e=222.05. (M+H).

Step C. 5,6-Diethyl-4-oxo-4,5-dihydro-3H-pyrrolo[3,2-d]pyrimidine-7-carbonitrile 12 (II-B1)

This compound was prepared using the procedure described in Example 9, except starting with 3-amino-4-cyano-1,5-diethyl-1H-pyrrole-2-carboxylic acid methyl ester (0.20 g, 0.79 mmol) to provide the title compound (0.13 g, 76% yield) as a white powder. ¹HNMR (500 MHz, DMSO-d6) δ12.3 (br s, 1H), 7.89 (s, 1H), 4.37 (q, J=7.1 Hz, 2H), 2.84 (q, J=7.6 Hz, 2H), 1.25 (m, 6H) ppm. MS (ES+): m/e=217.03 (M+H).

Step D. 5,6-Diethyl-4-thioxo-4,5-dihydro-3H-pyrrolo[3,2-d]pyrimidine-7-carbonitrile 13 (II-B2)

This compound was prepared using the procedure described in Example 11, except starting with 5,6-diethyl-4-oxo-4,5-dihydro-3H-pyrrolo[3,2-d]pyrimidine-7-carbonitrile (0.05 g, 0.23 mmol) to provide the title compound (0.05 g, 86% yield) as a pale yellow solid. ¹HNMR (500 MHz, DMSO-d₆) δ13.6 (br s, 1H), 8.05 (s, 1H), 4.86 (q, J=7.0 Hz, 2H), 2.90 (q, J=7.6 Hz, 2H), 1.25 (m, 6H) ppm. MS (ES+): m/e=233.02 (M+H).

### Example 14

### 5,6-Diphenyl-4-thioxo-4,4a,5,7a-tetrahydro-3H-pyrrolo[3,2-d]pyrimidine-7-carbonitrile (II-B18)

Step A. (Benzoyl-phenylamino)acetic acid methyl ester

To a solution of benzanilide (1.0 g, 5.07 mmol) in dimethylformamide (12.5 mL) at room temperature under nitrogen was added 60% sodium hydride/mineral oil suspension (0.24 g, 6.08 mmol) and the reaction was stirred 0.5 hours. To the reaction was dropwise added methyl bromoacetate (0.53 mL, 5.58 mmol) and stirring was continued for 3 hours. The reaction was diluted with ethyl acetate, was washed with 10% potassium bisulfate, three portions of water and brine, was dried (sodium sulfate) and was evaporated. Purification by flash chromatography (SiO₂) eluted with 35:65 ethyl acetate:hexanes provided the title compound (1.06 g, 77% yield) as a colorless oil. ¹HNMR (500 MHz, CDCl₃) δ7.38 (d, J=7.8 Hz, 2H), 7.3-7.1 (m, 8H), 4.65 (s, 2H), 3.81 (s, 3H) ppm. MS (ES+): m/e=270.07 (M+H).

Step B. [(2,2-Dicyano-1-phenyl-vinyl)-phenyl-amino]acetic acid methyl ester

This compound was prepared using the procedure described in Example 2, Step A, except starting with (benzoyl-phenylamino)acetic acid methyl ester (0.53 g, 1.95 mmol) to provide the title compound (0.12 g, 19% yield) as an off-white solid. ¹HNMR (500 MHz, CDCl₃) δ7.3-7.0 (m, 10H), 5.0 (s, 2H), 3.57 (s, 3H) ppm. MS (ES+): m/e=318.07 (M+H).

Step C. 3-Amino-4-cyano-1,5-diphenyl-1H-pyrrole-2-carboxylic acid ethyl ester

A solution of [(2,2-dicyano-1-phenyl-vinyl)-phenyl-amino]acetic acid methyl ester (0.10 g, 0.30 mmol) in ethanol (5 mL) was treated with sodium ethoxide (0.02 g, 0.36 mmol) and stirred at reflux under nitrogen for 4 hours. The reaction was cooled, was diluted with water, was extracted with three portions of dichloromethane, was dried (sodium sulfate) and was evaporated. Purification by flash chromatography (SiO₂) eluted with 2:8 ethyl acetate:hexanes provided the title compound (0.09 g, 94% yield) as a white solid. ¹HNMR (500 MHz, CDCl₃) δ7.3-7.0 (m, 10H), 5.05 (br s, 2H), 4.0 (q, J=7.2 Hz, 2H), 1.93 (t, J=7.2 Hz, 3H) ppm. MS (ES+): m/e=332.08 (M+H).

Step D. 5,6-Diphenyl-4-oxo-4,5-dihydro-3H-pyrrolo[3,2-d]pyrimidine-7-carbonitrile (II-B17)

This compound was prepared using the procedure described in Example 9, except starting with 3-amino-4-cyano-1,5-diphenyl-1H-pyrrole-2-carboxylic acid ethyl ester 22 (0.09 g, 0.29 mmol) to provide the title compound (0.07 g, 77% yield) as an off-white solid. ¹HNMR (500 MHz, DMSO-d6) δ12.6 (br s, 1H), 8.23 (s, 1H), 7.53 (m, 10H) ppm.

Step E. 5,6-Diphenyl-4-thioxo-4,5-dihydro-3H-pyrrolo[3,2-d]pyrimidine-7-carbonitrile (II-B18)

This compound was prepared using the procedure described in Example 11, except starting with 5,6-diphenyl-4-oxo-4,5-dihydro-3H-pyrrolo[3,2-d]pyrimidine-7-carbonitrile (0.05 g, 0.17 mmol) to provide the title compound (0.05 g, 85% yield) as a pale yellow solid. ¹HNMR (500 MHz, DMSO-d₆) δ8.29 (s, 1H), 7.45 (m, 10H), 4.18 (br s, 1H) ppm. MS (ES+): m/e=329.04 (M+H).

### Example 15

### 5,6-Diisobutyl-4-oxo-4,5-dihydro-3H-pyrrolo[3,2-d]pyrimidine-7-carbonitrile (II-B29)

Step A. 2-(1-Isobutylamino-3-methyl-butylidene)-malonitrile

This compound was prepared using the procedure described in example 2, except starting with N-isobutyl-3-methyl-butyramide (3.64 g, 23 mmol) to provide the title compound (0.86 g, 18% yield) as a colorless oil. H-NMR (500 MHz, CDCl3) δ6.27 (br s, 2H), 3.16 (m, 2H), 2.48 (m, 2H), 2.07 (m, 1H), 1.92 (m, 1H), 1.08 (d, J=6.6 Hz, 6H), 1.01 (d, J=6.7 Hz, 6H) ppm. MS (ES+): m/e 206.11 (M+H).

Step B. 3-Amino-4-cyano-1,5-diisobutyl-1H-pyrrole-2-carboxylic acid methyl ester

This compound was prepared using the procedure described in example 13 Step B, except starting with 2-(1-isobutylamino-3-methyl-butylidene)-malonitrile (0.50 g, 2.44 mmol) to provide the title compound (0.32 g, 47% yield) as a yellow solid. ¹H-NMR (500 MHz, CDCl3) δ4.81 (br s, 2H), 3.77 (s, 5H), 2.47 (d, J=7.5 Hz, 2H), 1.92 (m, 2H), 0.89 (d, J=6.6 Hz, 6H), 0.77 (d, J=6.3 Hz, 6H) ppm. MS (ES+): m/e 278.14 (M+H). Analytical HPLC (C18 column): 3.682 minutes.

Step C. 5,6-Diisobutyl-4-oxo-4,5-dihydro-3H-pyrrolo[3,2-d]pyrimidine-7-carbonitrile

This compound was prepared using the procedure described in example 9, except starting with 3-amino-4-cyano-1,5-diisobutyl-1H-pyrrole-2-carboxylic acid methyl ester (0.31 g, 1.1 mmol) to provide the title compound (0.17 g, 59% yield) as an off-white solid. ¹H-NMR (500 MHz, DMSO-d6) δ12.1 (s, 1H), 7.76 (d, J=0.9 Hz, 1H), 4.02 (s, 2H), 2.57 (d, J=7.4 Hz, 2H), 1.86 (m, 2H), 0.75 (d, J=6.5 Hz, 6H), 0.63 (d, J=6.6 Hz, 6H) ppm. MS (ES+): m/e 273.10 (M+H). Analytical HPLC (C18 column): 3.225 minutes.

### Example 16

### [2-(7-Cyano-5-ethyl-4-oxo-4,5-dihydro-3H-pyrrolo[3,2-d]pyrimidin-6-yl)-ethyl]-carbamic acid benzyl ester (II-B30)

Step A. (4,4-Dicyano-3-ethylamino-but-3-enyl)-carbamic acid benzyl ester

This compound was prepared using the procedure described in Example 2 Step A, except starting with (2-ethylcarbamoyl-ethyl)-carbamic acid benzyl ester (1.26 g, 5.0 mmol) to provide the title compound (0.35 g, 24% yield) as a colorless oil. ¹H-NMR (500 MHz, CDCl3) δ7.4 (m, 5H), 6.4 (br s, 1H), 5.4 (br s, 1H), 5.1 (s, 2H), 3.55 (m, 2H), 3.45 (m, 2H), 2.85 (m, 2H), 1.30 (m, 3H) ppm. MS (ES+): m/e 299.10 (M+H).

Step B. 3-Amino-5-(2-benzyloxycarbonylamino-ethyl)-4-cyano-1-ethyl-1H-pyrrole-2-carboxylic acid methyl ester

This compound was prepared using the procedure described in example 13 Step B, except starting with (4,4-dicyano-3-ethylamino-but-3-enyl)-carbamic acid benzyl ester (0.54 g, 1.81 mmol) to provide the title compound (0.34 g, 51% yield) as a colorless glassy solid. MS (ES+): m/e 371.20 (M+H). Analytical HPLC (C18 column): 3.279 minutes (and impurities).

Step C. [2-(7-Cyano-5-ethyl-4-oxo-4,5-dihydro-3H-pyrrolo[3,2-d]pyrimidin-6-yl)-ethyl]-carbamic acid benzyl ester (II-B30)

This compound was prepared using the procedure described in Example 9, except starting with 3-amino-5-(2-benzyloxycarbonylamino-ethyl)-4-cyano-1-ethyl-1H-pyrrole-2-carboxylic acid methyl ester (0.50 g, 1.38 mmol) to provide the title compound (0.22 g, 44% yield) as a white solid. ¹H-NMR (500 MHz, DMSO-d6) δ12.5 (s, 1H), 8.13 (s, 1H), 7.68 (m, 1H), 7.32 (m, 4H), 5.17 (s, 2H), 4.56 (m, 2H), 3.40 (m, 2H), 3.21 (m, 2H), 1.48 (t, J=6.9 Hz, 3H) ppm, MS (ES+): m/e 366.21 (M+H). Analytical HPLC (C18 column): 2.864 minutes. IR: 2226.7, 1681.5, 1589.6 cm⁻¹.

### Example 17

### [2-(7-Cyano-5-ethyl-4-thioxo-4,5-dihydro-3H-pyrrolo[3,2-d]pyrimidin-6-yl)-ethyl]-carbamic acid benzyl ester (II-B31)

This compound was prepared using the procedure described in Example 11, except starting with [2-(7-cyano-5-ethyl-4-oxo-4,5-dihydro-3H-pyrrolo[3,2-d]pyrimidin-6-yl)-ethyl]-carbamic acid benzyl ester (0.10 g, 0.26 mmol) to provide the title compound (0.03 g, 28% yield) as a pale yellow solid. ¹H-NMR (500 MHz, DMSO-d6) δ13.7 (s, 1H), 8.13 (s, 1H), 7.52 (m, 1H), 7.32 (m, 5H), 5.00 (s, 2H), 4.90 (m, 2H), 3.40 (m, 2H), 3.11 (m, 2H), 1.32 9M, 3H) ppm, MS (ES+): m/e 382.15 (M+H). Analytical HPLC (C18 column): 3.169 minutes. IR: 2226.7, 1665.3, 1585.0, 1534.5 cm⁻¹.

### Example 18

### 6-Methylsulfanyl-4-thioxo-3,4-dihydro-thieno[3,2-d]pyrimidine-7-carbonitrile (III-5)

Step A. 6-Methylsulfanyl-4-oxo-3,4-dihydro-thieno[3,2-d]pyrimidine-7-carbonitrile (III-4)

Malononitrile (5 mmol) was added to a suspension of K₂CO₃ (2.1 g, 15 mmol) in DMF (4.5 mL). After 10 minutes, CS₂ (7.5 mmol) was added in one portion and the resulting mixture was stirred at room temperature for an additional 10 minutes. A solution of 1-chloro-acetamide (5 mmol) in DMF (5 mL) was added with cooling and after 1 hour, a solution of MeI (5.5 mmol) in DMF (2 mL) was added dropwise. After 30 minutes, the mixture was poured onto water (90 mL) and the resulting mixture was stirred vigorously for 16 hours to afford a suspension of crude intermediate 3-amino-4-cyano-5-methylsulfanyl-thiophene-2-carboxylic acid amide. This crude product was filtered off and washed extensively with water and small amount of cold methanol to provide crude intermediate (0.5 g, 46% yield). LC−MS (ES+) 213.9 (M+H).

The crude intermediate (100 mg, 0.47 mmol) and DMF-DMA (0.56 mmol) were mixed in acetonitrile (3 mL) and heated at 90° C. for 3 hours. The reaction mixture was concentrated to provide 4-cyano-3-(dimethylamino-methyleneamino)-5-methylsulfanyl-thiophene-2-carboxylic acid amide which was used directly in the next step. This crude amide was dissolved in glacial acetic acid (3 mL), and the resulting mixture was heated to 90° C. for 30 minutes. The reaction mixture was concentrated then, the reaction mixture was washed with a small amount of ethyl acetate and ether and dried in vacuo. 6-methylsulfanyl-4-oxo-3,4-dihydro-thieno[3,2-c]pyrimidine-7-carbonitrile (Compound III-4) was obtained without further purification (75 mg, 71%). ¹HNMR (500 MHz, DMSO-d6) δ8.3 (2, 1H), 3.3 (s, 1H), 2.85 (s, 3H). LC−MS (ES+): m/e=223.9 (M+H).

Step B. 6-Methylsulfanyl-4-thioxo-3,4-dihydro-thieno[3,2-d]pyrimidine-7-carbonitrile (III-5)

Compound III-4 (30 mg, 0.135 mmol ) was dissolved in toluene (1.5 mL) and Lawesson reagent (0.161 mmol) was added and the reaction mixture was heated to reflux for 18 hours. The reaction mixture was concentrated and then after the aqueous work-up, the product was purified by preparatory HPLC to afford the title compound (4.5 mg, 13%). LC−MS (ES+): m/e=239.9 (M+H)

### Example 19

### 6-Isopropylamino-4-oxo-3,4-dihydro-thieno[3,2-d]pyrimidine-7-carbonitrile (III-21)

Step A. 6-Methanesulfonyl-4-oxo-3,4-dihydro-thieno[3,2-d]pyrimidine-7-carbonitrile (III-6)

To compound III-4 (100 mg, 0.44 mmol) in dichloromethane (4 mL) was added m-CPBA (3 equivalents) and the reaction mixture was stirred at room temperature for 5 hours. The solid precipitate was filtered and washed extensively with dichloromethane to give the crude compound (III-6).

Step B. 6-Isopropylamino-4-oxo-3,4-dihydro-thieno[3,2-d]pyrimidine-7-carbonitrile (III-21)

The crude product III-6 (50 mg, 0.2 mmol) and isopropylamine (3 equivalents) were mixed in 2 mL acetonitrile and heated at 70° C. for 18 hours. The solid precipitate was filtered off and washed with a small amount of acetonitrile and washed with dichloromethane to give compound III-21 without further purification (50% yield). ¹HNMR (500 MHz, DMSO-d₆) δ1.24 (d, 6H), 3.7 (m, 1H), 8.1 (s, 1H), 9.8 (s, 1H). LC−MS (ES+): m/e=235.0 (M+H)

### Example 20

### 6-Propylamino-4-oxo-3,4-dihydro-thieno [3,2-d]pyrimidine-7-carbonitrile (Compound III-22)

This compound was prepared using the procedure described in Example 19 except starting with propylamine to provide compound III-22 (63% yield). ¹HNMR(500 MHz, DMSO-d₆) δ0.9 (t, 3H), 1.6 (m, 2H), 3.25 (t, 2H), 8.1 (s, 1H), 8.85 (broad peak, 1H). LC−MS (ES+): m/e=235.0 (M+H).

### Example 21

### 6-Isobutylamino-4-oxo-3,4-dihydro-thieno[3,2-d]pyrimidine-7-carbonitrile (III-23)

This compound was prepared using the procedure described in Example 19 except starting with isobutylamine to provide the compound III-23 (45% yield). ¹HNMR(500 MHz, DMSO-d₆) δ0.9 (d, 6H), 3.05 (m, 2H), 1.95 (m, 1H), 8.1 (s, 1H). LC−MS (ES+): m/e=249.0 (M+H).

### Example 22

### 6-Benzylamino-4-oxo-3,4-dihydro-thieno [3,2-d]pyrimidine-7-carbonitrile (III-26)

This compound was prepared using the procedure described in Example 19 except starting with benzylamine to provide the compound III-26 (70% yield). ¹HNMR(500 MHz, DMSO-d₆) δ4.52 (S, 2H), 7.4(m, 5H), 8.1 (s, 1H). LC−MS (ES+): m/e=283.0 (M+H).

### Example 23

### 6-Cyclopentylamino-4-oxo-3,4-dihydro-thieno[3,2-d]pyrimidine-7-carbonitrile (III-24)

This compound was prepared using the procedure described in Example 19 except starting with cyclopentylamine to provide the compound III-24 (42% yield). ¹HNMR(500 MHz, DMSO-d₆) δ1.6 (m, 6H), 2.0 (m, 2H), 3.9(m, 1H), 8.1 (s, 1H). LC−MS (ES+): m/e=261.0 (M+H).

### Example 24

### 6-Cyclohexylamino-4-oxo-3,4-dihydro-thieno[3,2-d]pyrimidine-7-carbonitrile (III-25)

This compound was prepared using the procedure described in Example 19 except starting with cyclohexylamine to provide the compound III-25 (47% yield). LC−MS (ES+): m/e=261.0 (M+H).

### Example 25

### 10-(2H-Tetrazol-5-yl)-6,7,8,9-tetrahydro-3H,5H-1,3,4b-triaza-benzo[a]azulene-4-thione (II-A28)

4-Thioxo-4,5,6,7,8,9-hexahydro-3H-1,3,4b-triaza-benzo[a]azulene-10-carbonitrile (65 mg, 26 mmol) was suspended in 10 mL dry THF, AlCl₃ (36 mg, 26 mmol) and NaN₃ (76 mg, 12 mmol) were added. The solution was heated to reflux under N₂ for 96 hours. The reaction was cooled to room temperature and acidified to pH 3 using 2N HCl. The acidic solution was evaporated to yield 40 mg of solid material. This was purified by HPLC, using a gradient of 10-100% 0.1% TFA and acetonitrile/water over 15 minutes, to yield 15 mg (20%) of the desired product. ¹H NMR (500 MHz, DMSO-d6): 13.38 (s, 1H), 7.95 (s, 1H), 5.30 (s, 2H), 3.25 (s, 2H), 3.15 (s, 1H), 1.68 (s, 2H), 1.50 (s, 4H). MS (M+H) 288.06.

### Example 26

### 4-Thioxo-4,5,6,7,8,9-hexahydro-3H-1,3,4b-triaza-benzo[a]azulene-10-carboxylic acid amide (II-A82)

To 4-thioxo-4,5,6,7,8,9-hexahydro-3H-1,3,4b-triaza-benzo[a]azulene-10-carbonitrile (100 mg, 0.41 mmol) was added 5N NaOH (3 mL) and the turbid suspension was heated to 100° C. After 14 hours, the reaction mixture was poured into water, cooled to 5° C., and acidified with acetic acid to pH5. This resulted in a pale yellow precipitate that was collected by filtration and dried under vacuum to give the title compound (87 mg, 81% yield). ¹HNMR(500 MHz, DMSO-d₆) δ13.55-13.35 (1H, bs), 8.15 (1H, s), 8.05 (1H, s), 7.4 (1H, s), 5.55-5.35 (2H, m) 3,60-3.50 (2H, m), 1.85-1.65 (2H, m), 1.60-1.50 (4H, m); MS (m/z) 263.03 (M+H)

### Example 27

### 5,6-Diisobutyl-4-oxo-4,5-dihydro-3H-pyrrolo[3,2-d]pyrimidine-7-carbonitrile (II-B29)

Step A. 2-(1-Isobutylamino-3-methyl-butylidene)-malonitrile

This compound was prepared using the procedure described in Example 2, except starting with N-isobutyl-3-methyl-butyramide (3.64 g, 23 mmol) to provide the title compound (0.86 g, 18% yield) as a colorless oil. ¹H-NMR (500 MHz, CDCl3) δ6.27 (br s, 2H), 3.16 (m, 2H), 2.48 (m, 2H), 2.07 (m, 1H), 1.92 (m, 1H), 1.08 (d, J=6.6 Hz, 6H), 1.01 (d, J=6.7 Hz, 6H) ppm. MS (ES+): m/e 206.11 (M+H).

Step B. 3-Amino-4-cyano-1,5-diisobutyl-1H-pyrrole-2-carboxylic acid methyl ester

This compound was prepared using the procedure described in example 13 Step B, except starting with 2-(1-isobutylamino-3-methyl-butylidene)-malonitrile (0.50 g, 2.44 mmol) to provide the title compound (0.32 g, 47% yield) as a yellow solid. ¹H-NMR (500 MHz, CDCl3) δ4.81 (br s, 2H), 3.77 (s, 5H), 2.47 (d, J=7.5 Hz, 2H), 1.92 (m, 2H), 0.89 (d, J=6.6 Hz, 6H), 0.77 (d, J=6.3 Hz, 6H) ppm. MS (ES+): m/e 278.14 (M+H). Analytical HPLC (C18 column): 3.682 minutes.

Step C. 5,6-Diisobutyl-4-oxo-4,5-dihydro-3H-pyrrolo[3,2-d]pyrimidine-7-carbonitrile

This compound was prepared using the procedure described in example 9, except starting with 3-amino-4-cyano-1,5-diisobutyl-1H-pyrrole-2-carboxylic acid methyl ester (0.31 g, 1.1 mmol) to provide the title compound (0.17 g, 59% yield) as an off-white solid. ¹H-NMR (500 MHz, DMSO-d6) δ12.1 (s, 1H), 7.76 (d, J=0.9 Hz, 1H), 4.02 (s, 2H), 2.57 (d, J=7.4 Hz, 2H), 1.86 (m, 2H), 0.75 (d, J=6.5 Hz, 6H), 0.63 (d, J=6.6 Hz, 6H) ppm. MS (ES+): m/e 273.10 (M+H). Analytical HPLC (C18 column): 3.225 minutes.

### Example 28

### [2-(7-Cyano-5-ethyl-4-oxo-4,5-dihydro-3H-pyrrolo[3,2-d]pyrimidin-6-yl)-ethyl]-carbamic acid benzyl ester (II-B30)

Step A. (4,4-Dicyano-3-ethylamino-but-3-enyl)-carbamic acid benzyl ester

This compound was prepared using the procedure described in Example 2 Step A, except starting with (2-ethylcarbamoyl-ethyl)-carbamic acid benzyl ester (1.26 g, 5.0 mmol) to provide the title compound (0.35 g, 24% yield) as a colorless oil. ¹H-NMR (500 MHz, CDCl3) δ7.4 (m, 5H), 6.4 (br s, 1H), 5.4 (br s, 1H), 5.1 (s, 2H), 3.55 (m, 2H), 3.45 (m, 2H), 2.85 (m, 2H), 1.30 (m, 3H) ppm. MS (ES+): m/e 299.10 (M+H).

Step B. 3-Amino-5-(2-benzyloxycarbonylamino-ethyl)-4-cyano-1-ethyl-1H-pyrrole-2-carboxylic acid methyl ester

This compound was prepared using the procedure described in example 13 Step B. except starting with (4,4-dicyano-3-ethylamino-but-3-enyl)-carbamic acid benzyl ester (0.54 g, 1.81 mmol) to provide the title compound (0.34 g, 51% yield) as a colorless glassy solid. MS (ES+): m/e 371.20 (M+H). Analytical HPLC (C18 column): 3.279 minutes (and impurities).

Step C. [2-(7-Cyano-5-ethyl-4-oxo-4,5-dihydro-3H-pyrrolo[3,2-d]pyrimidin-6-yl)-ethyl]-carbamic acid benzyl ester (II-B30)

This compound was prepared using the procedure described in example 9, except starting with 3-amino-5-(2-benzyloxycarbonylamino-ethyl)-4-cyano-1-ethyl-1H-pyrrole-2-carboxylic acid methyl ester (0.50 g, 1.38 mmol) to provide the title compound (0.22 g, 44% yield) as a white solid. ¹H-NMR (500 MHz, DMSO-d6) δ12.5 (s, 1H), 8.13 (s, 1H), 7.68 (m, 1H), 7.32 (m, 4H), 5.17 (s, 2H), 4.56 (m, 2H), 3.40 (m, 2H), 3.21 (m, 2H), 1.48 (t, J=6.9 Hz, 3H) ppm, MS (ES+): m/e 366.21 (M+H). Analytical HPLC (C18 column): 2.864 minutes. IR: 2226.7, 1681.5, 1589.6 cm⁻¹.

### Example 29

### [2-(7-Cyano-5-ethyl-4-thioxo-4,5-dihydro-3H-pyrrolo [3,2-d]pyrimidin-6-yl)-ethyl]-carbamic acid benzyl ester (II-B31)

This compound was prepared using the procedure described in example 11, except starting with [2-(7-cyano-5-ethyl-4-oxo-4,5-dihydro-3H-pyrrolo[3,2-d]pyrimidin-6-yl)-ethyl]-carbamic acid benzyl ester (0.10 g, 0.26 mmol) to provide the title compound (0.03 g, 28% yield) as a pale yellow solid. ¹H-NMR (500 MHz, DMSO-d6) δ13.7 (s, 1H), 8.13 (s, 1H), 7.52 (m, 1H), 7.32 (m, 5H), 5.00 (s, 2H), 4.90 (m, 2H), 3.40 (m, 2H), 3.11 (m, 2H), 1.32 9M, 3H) ppm, MS (ES+): m/e 382.15 (M+H). Analytical HPLC (C18 column): 3.169 minutes. IR: 2226.7, 1665.3, 1585.0, 1534.5 cm⁻¹.

### Example 30

### 6-(2-Amino-ethyl)-5-ethyl-4-oxo-4,5-dihydro-3H-pyrrolo[3.2-d]pyrimidine-7-carbonitrile (II-B27)

A solution of [2-(7-cyano-5-ethyl-4-oxo-4,5-dihydro-3H-pyrrolo[3,2-d]pyrimidin-6-yl)-ethyl]-carbamic acid benzyl ester (0.02 g, 0.06 mmol) in methanol (3 mL) was treated with Pd(OH)₂ (0.01 g) and stirred under hydrogen (1 atm) for 1 hour. The reaction was filtered through Celite, evaporated and purified by flash chromatography (SiO₂) eluted with 2:8 methanol:dichloromethane to provide the title compound (0.01 g, 69% yield) as a white solid. ¹H-NMR (500 MHz, CD₃OD) d 7.70 (s, 1H), 4.30 (m, 2H), 3.84 (m, 2H), 2.71 (m, 2H), 1.24 (t, J=6.8 Hz, 3H) ppm. Analytical HPLC (C18 column): 0.25 minutes.

### Example 31

### 5-Ethyl-4-oxo-6-phenyl-4,5-dihydro-3H-pyrrolo[3,2-d]pyrimidine-7-carbonitrile (II-B32)

Step A. 2-(Ethylamino-phenyl-methylene)-malonitrile

This compound was prepared using the procedure described in Example 2 Step A, except starting N-ethylbenzamide (3.43 g, 23.0 mmol) to provide the title compound (1.12 g, 25% yield) as a white solid. ¹H-NMR (500 MHz, CDCl₃) δ7.2-7.6 (m, 5H), 6.6 (br s, 1H), 5.4 (br s, 1H), 3.09 (m, 2H), 1.07 (t, J=7.2 Hz, 3H) ppm. MS (ES+): m/e 198.04 (M+H). Analytical HPLC (C18 column): 2.882 minutes.

Step B. 3-Amino-4-cyano-1-ethyl-5-phenyl-1H-pyrrole-2-carboxylic acid methyl ester

This compound was prepared using the procedure described in example 13 Step B, except starting with 2-(ethylamino-phenyl-methylene)-malonitrile (0.50 g, 2.53 mmol) to provide the title compound (0.60 g, 89% yield) as a white solid. ¹H-NMR (500 MHz, CDCl₃) δ7.45 (m, 3H), 7.35 (m, 2H), 4.90 (s, 2H), 4.12 (m, 2H), 3.80 (m, 2H), 1.10 (m, 3H) ppm. MS (ES+): m/e 270.11 (M+H). Analytical HPLC (C18 column): 3.381 minutes.

Step C. 5-Ethyl-4-oxo-6-phenyl-4,5-dihydro-3H-pyrrolo[3,2-d]pyrimidine-7-carbonitrile (II-B32)

This compound was prepared using the procedure described in example 9, except starting with 3-amino-4-cyano-1-ethyl-5-phenyl-1H-pyrrole-2-carboxylic acid methyl ester (0.60 g, 2.21 mmol) to provide the title compound (0.07 g, 13% yield) as a white solid. ¹H-NMR (500 MHz, DMSO-d6) δ12.5 (s, 1H), 8.06 (s, 1H), 7.65 (s, 5H), 4.36 (q, J=7. Hz, 2H), 1.23 (t, J=7.1 Hz, 3H) ppm, MS (ES+): m/e 265.06 (M+H). Analytical HPLC (C18 column): 2.930 minutes.

### Example 32

### 5-Ethyl-6-phenyl-4-thioxo-4,5-dihydro-3H-pyrrolo[3,2-d]pyrimidine-7-carbonitrile (II-B26)

This compound was prepared using the procedure described in example 11, except starting with 5-ethyl-4-oxo-6-phenyl-4,5-dihydro-3H-pyrrolo[3,2-d]pyrimidine-7-carbonitrile (0.05 g, 0.17 mmol) to provide the title compound (0.01 g, 30% yield) as a yellow solid. ¹H-NMR (500 MHz, DMSO-d6) δ13.6 (s, 1H), 8.00 (s, 1H), 7.46 (s, 5H), 4.60 (q, J=6.7 Hz, 2H), 1.32 (t, J=6.7 Hz, 3H) ppm, MS (ES+): m/e 281.07 (M+H). Analytical HPLC (C18 column): 3.289 minutes.

### Example 33

### 6-Piperidin-4-oxo-3,4-dihydro-thieno [3,2-d]pyrimidine-7-carbonitrile (III-33)

This compound was prepared using the procedure described in Example 19 except starting with piperidine to provide the title compound in 42% yield. LC−MS (ES+): m/e=261.0 (M+H).

### Example 34

### 6-Cyclopropylamino-4-oxo-3,4-dihydro-thieno[3,2-d]pyrimidine-7-carbonitrile (III-34)

This compound was prepared using the procedure described in Example 19 except starting with cyclopropylamine to provide the title compound in 42% yield. LC−MS (ES+): m/e=233.0 (M+H).

### Example 35

### 6-Cyclohexylmethylamino-4-oxo-3,4-dihydro-thieno[3,2-d]pyrimidine-7-carbonitrile (III-35)

This compound was prepared using the procedure described in Example 19 except starting with cyclohexylmethylamine in place of isopropylamine to provide the title compound in 42% yield. LC−MS (ES+): m/e=289.1 (M+H).

### Example 36

### 6-(3-Methyl-butylamino)-4-oxo-3,4-dihydro-thieno[3,2-d]pyrimidine-7-carbonitrile (III-36)

This compound was prepared using the procedure described in Example 19 except starting with 6-(3-Methyl-butylamino)- to provide the compound III-36 (42% yield). LC−MS (ES+): m/e=263.1 (M+H).

### Example 37

### 6-[2-(1H-Imidazol-4-yl)-ethylamino]-4-oxo-3,4-dihydro-thieno[3,2-d]pyrimidine-7-carbonitrile (III-37)

This compound was prepared using the procedure described in Example 19 except starting with 6-[2-(1H-imidazol-4-yl)-ethylamine to provide the title compound in 42% yield. LC−MS (ES+): m/e=287.0 (M+H).

### Example 38

### 6-Ethyl amine-4-oxo-3,4-dihydro-thieno [3,2-d]pyrimidine-7-carbonitrile (III-38)

This compound was prepared using the procedure described in Example 19 except starting with ethylamine to provide the title compound in 42% yield. LC−MS (ES+): m/e=221.0 (M+H).

### Example 39

### 6-(Methyl-propyl-amino)-4-oxo-3,4-dihydro-thieno[3,2-d]pyrimidine-7-carbonitrile (III-39)

This compound was prepared using the procedure described in Example 19 except starting with N-methyl-propylamine to provide the title compound in 42% yield. LC−MS (ES+): m/e=249.0 (M+H).

### Biological Methods

### IC₅₀ Determination for the Inhibition of GSK-3

Compounds were screened for their ability to inhibit GSK-3β (AA 1-420) activity using a standard coupled enzyme system (Fox et al. (1998) Protein Sci. 7, 2249). Reactions were carried out in a solution containing 100 mM HEPES (pH 7.5), 10 mM MgCl₂, 25 mM NaCl, 300 μM NADH, 1 mM DTT and 1.5% DMSO. Final substrate concentrations in the assay were 10 μM ATP (Sigma Chemicals, St Louis, Mo.) and 300 μM peptide (HSSPHQS(PO₃H₂)EDEEE, American Peptide, Sunnyvale, Calif.). Reactions were carried out at 30° C. and 60 nM GSK-3β. Final concentrations of the components of the coupled enzyme system were 2.5 mM phosphoenolpyruvate, 300 μM NADH, 30 μg/ml pyruvate kinase and 10 μg/ml lactate dehydrogenase.

An assay stock buffer solution was prepared containing all of the reagents listed above with the exception of ATP and the test compound of interest. 59 μl of the test reaction was placed in a 96 well ½ diameter plate (Corning, Corning, N.Y.) then treated with 1 μl of a 2 mM DMSO stock containing the test compound (final compound concentration 30 μM). The plate was incubated for 10 minutes at 30° C. then the reaction initiated by addition of 7 μl of ATP (final concentration 10 μM). Rates of reaction were obtained using a Molecular Devices Spectramax plate reader (Sunnyvale, Calif.) over a 5 minute read time at 30° C. Compounds showing greater than 50% inhibition versus standard wells containing DMSO, but no compound, were titrated and IC₅₀ values were determined using a similar protocol in standard 96 well plates with the assay scaled to a final volume of 200 μl.

In the GSK-3 inhibition assay described above, many of the compounds of this invention that were tested were found to provide an IC₅₀ value below one micromolar.

### Kᵢ Determination for the Inhibition of GSK-3

Compounds were screened for their ability to inhibit GSK-3β (AA 1-420) activity using a standard coupled enzyme system (Fox et al. (1998) Protein Sci. 7, 2249). Reactions were carried out in a solution containing 100 mM HEPES (pH 7.5), 10 mM MgCl₂, 25 mM NaCl, 300 μM NADH, 1 mM DTT and 1.5% DMSO. Final substrate concentrations in the assay were 20 μM ATP (Sigma Chemicals, St Louis, Mo.) and 300 μM peptide (HSSPHQS(PO₃H₂)EDEEE, American Peptide, Sunnyvale, Calif.). Reactions were carried out at 30° C. and 20 nM GSK-3β. Final concentrations of the components of the coupled enzyme system were 2.5 mM phosphoenolpyruvate, 300 μM NADH, 30 μg/ml pyruvate kinase and 10 μg/ml lactate dehydrogenase.

An assay stock buffer solution was prepared containing all of the reagents listed above with the exception of ATP and the test compound of interest. The assay stock buffer solution (175 μl) was incubated in a 96 well plate with 5 μl of the test compound of interest at final concentrations spanning 0.002 μM to 30 μM at 30° C. for 10 minutes. Typically, a 12 point titration was conducted by preparing serial dilutions (from 10 mM compound stocks) with DMSO of the test compounds in daughter plates. The reaction was initiated by the addition of 20 μl of ATP (final concentration 20 μM). Rates of reaction were obtained using a Molecular Devices Spectramax plate reader (Sunnyvale, Calif.) over 10 minutes at 30° C. The Kᵢ values were determined from the rate data as a function of inhibitor concentration.

In the GSK-3 inhibition assay described above, many of the compounds of this invention that were tested were found to provide a Kᵢ value below one micromolar.

### Rock Inhibition Assay

Compounds were screened for their ability to inhibit ROCK using a standard coupled enzyme assay (Fox et al (1998)Protein Sci 7, 2249). Reactions were carried out in 100 mM HEPES pH 7.5, 10 mM MgCl2, 25 mM NaCl, 1 mM DTT and 1.5% DMSO. Final substrate concentrations in the assay were 13 μM ATP (Sigma chemicals) and 200 μM peptide (KKRNRTLSV, American Peptide, Sunnyvale, Calif.). Assays were carried out at 30° C. and 200 nM ROCK. Final concentrations of the components of the coupled enzyme system were 2.5 mM phosphoenolpyruvate, 400 μM NADH, 30 μg/ml pyruvate kinase and 10 μg/ml lactate dehydrogenase.

An assay stock buffer solution was prepared containing all of the reagents listed above, with the exception of ROCK, DTT and the test compound of interest. 56 μl of the test reaction was placed in a 384 well plate followed by addition of 1 μl of 2 mM DMSO stock containing the test compound (final compound concentration 30 μM). The plate was preincubated for ˜10 minutes at 30° C. and the reaction initiated by addition of 10 μl of enzyme (final concentration 100 nM). Rates of reaction were obtained using a BioRad Ultramark plate reader (Hercules, Calif.) over a 5 minute read time at 30° C. Compounds showing >50% inhibition versus standard wells containing DMSO, but no compound, were titrated and IC50's determined using a similar protocol.

In the ROCK inhibition assay described above, certain compounds of this invention were tested and were found to inhibit ROCK kinase.

While we have described a number of embodiments of this invention, it is apparent that our basic examples may be altered to provide other embodiments that utilize the compounds and methods of this invention. Therefore, it will be appreciated that the scope of this invention is to be defined by the appended claims rather than by the specific embodiments that have been represented by way of example.

We claim:

1. A compound of formula I: 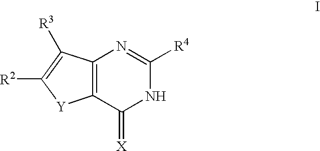
or a pharmaceutically acceptable derivative thereof, wherein:
X is oxygen or sulfur;
Y is —S—, —O—, or —NR¹—;
R¹ is selected from R, CO₂R, C(O)R, CON(R)₂, SO₂R, SO₂N(R)₂, or an optionally substituted 5-7 membered monocyclic or 8-10 membered bicyclic saturated, partially unsaturated, or fully unsaturated ring having 0-3 heteroatoms independently selected from nitrogen, oxygen, or sulfur;
each R is independently selected from hydrogen or an optionally substituted C₁₋₆ aliphatic group;
R² is selected from R, N(R)₂, OR, SR, C(O)R, CO₂R, C(O)N(R)₂, NRN(R)₂, NRCOR, NRCO₂(C₁₋₆ aliphatic), NRSO₂(C₁₋₆ aliphatic), S(O)(C₁-₆ aliphatic), SO₂R, SO₂N(R)₂, or an optionally substituted 5-7 membered monocyclic or 8-10 membered bicyclic saturated, partially unsaturated, or fully unsaturated ring having 0-3 heteroatoms independently selected from nitrogen, oxygen, or sulfur, or:
(a) when Y is —NR¹—, R¹ and R² are taken together to form a saturated, partially unsaturated, or fully unsaturated 4-9 membered mono- or bicyclic ring having 1-2 heteroatoms, in addition to the —NR¹— nitrogen, independently selected from nitrogen, oxygen, or sulfur, wherein said ring formed by R¹ and R² is optionally substituted with 1-2 R⁶; or
(b) R² and R³ are taken together to form a saturated, partially unsaturated, or fully unsaturated 5-9 membered mono- or bicyclic ring having 0-2 heteroatoms independently selected from nitrogen, oxygen, or sulfur, wherein said ring formed by R² and R³ is optionally substituted with 1-2 R⁶;
R³ is selected from R, CN, halogen, NO₂, or Q₍ₙ₎R⁵, wherein:
n is selected from zero or one;
Q is a C₁₋₄ straight or branched alkylidene chain, wherein up to two non-adjacent methylene units of Q are optionally and independently replaced by O, S, NR, C(O), CO₂, CONR, OC(O)NR, NRCO, NRCO₂, NRCONR, S(O), SO₂, NRSO₂, or SO₂NR;
R⁴ is selected from R, N(R)₂, NRCOR, NRCO₂R, or an optionally substituted 5-7 membered monocyclic or 8-10 membered bicyclic saturated, partially unsaturated, or fully unsaturated ring having 0-3 heteroatoms independently selected from nitrogen, oxygen, or sulfur;
R⁵ is selected from R or an optionally substituted 5-14 membered mono-, bi-, or tricyclic aromatic, partially unsaturated, or saturated ring having 0-4 heteroatoms independently selected from nitrogen, oxygen, or sulfur; and
each R⁶ is independently selected from R, oxo, halogen, CN, C(O)R, CO₂R, SO₂R, OR, SR, N(R)₂, NRC(O)R, C(O)N(R)₂, NRCO₂R, OC(O)N(R)₂, NRSO₂R, or SO₂NR.

2. The compound according to claim 1, wherein:
Y is —NR¹—, and said compound has one or more features selected from the group consisting of:
(a) R¹ is selected from R, C(O)R, C(O)N(R)₂, SO₂R, CO₂R, or an optionally substituted 5-6 membered saturated, partially unsaturated, or fully unsaturated ring having 0-2 heteroatoms independently selected from nitrogen, oxygen, or sulfur;
(b) R² is selected from R, N(R)₂, OR, SR, C(O)R, CO₂R, C(O)N(R)₂, NRN(R)₂, NRC(O)R, SO₂R, or an optionally substituted 5-7 membered saturated, partially unsaturated, or fully unsaturated ring having 0-2 heteroatoms independently selected from nitrogen, oxygen, or sulfur, or R² and R¹ are taken together to form an optionally substituted 5-8 membered saturated, partially unsaturated, or aromatic ring having 0-1 heteroatoms, in addition to the nitrogen of R¹, independently selected from nitrogen, oxygen, or sulfur;
(c) R³ is selected from R, CN, or Q₍ₙ₎R⁵, wherein n is zero or one, Q is selected from a C₁₋₄ alkylidene chain wherein one methylene unit of Q is optionally replaced by O, S, NR, C(O), CO₂, CONR, NRC(O), NRC(O)NR, SO₂, or NRSO₂, and R⁵ is selected from R or an optionally substituted 5-7 membered saturated, partially unsaturated, or fully unsaturated ring having 0-4 heteroatoms independently selected from nitrogen, oxygen, or sulfur; and
(d) R⁴ is selected from R, N(R)₂, or an optionally substituted 5-6 membered saturated, partially unsaturated, or fully unsaturated ring having 0-2 heteroatoms independently selected from nitrogen, oxygen, or sulfur.

3. The compound according to claim 2, wherein:
R¹ is selected from hydrogen, methyl, ethyl, i-propyl, i-butyl, phenyl, CH₂CH₂(morpholin-4-yl), CH₂CH₂phenyl, CH₂phenyl, COMe, CONH₂, CH₂CONH₂, SC₂Me, CH₂SO₂NH₂, CO₂Et, or cyclopropyl;
R² is selected from hydrogen, methyl, ethyl, i-propyl, i-butyl, CF₃, phenyl, CH₂CH₂NH₂, NH₂, NHC(O)CH₃, CH₂CH₂NHC(O)OCH₂phenyl, SCH₃, SO₂CH₃, NHCH₃, SEt, CH₂phenyl, Oi-propyl, morpholin-4-yl, piperidin-1-yl, 4-methyl-piperazin-1-yl, thiomorpholin-4-yl, pyrrolidin-1-yl, thiazol-3-yl, oxazol-3-yl, azepan-1-yl, N(Me)₂, NHi-propyl, NHpropyl, NHi-butyl, NH-cyclopentyl, NH-cyclohexyl, NHCH₂phenyl, NHSO₂CH₃, NHNH₂, N(Me)propyl, NH-cyclopropyl, NHCH₂cyclohexyl, NHCH₂CH₂CH(CH₃)₂, or NHCH₂CH₂imidazol-4-yl;
R³ is selected from hydrogen, CN, CO₂H, CH₂CN, methyl, CH₂CONH₂, CH₂CO₂CH₃, —C≡CH, C(O)CH₃, CH₂CH₂CN, CH₂CH₂CH₂NH₂, hydrogen, CH₂CO₂H, CO₂Et, CH₂SO₂CH₃, CH₂NHSO₂CH₃, C(O)NH₂, CH₂NHC(O)CH₃, CH₂CH₂OH, C(O)CH₂CH₃, oxadiazolyl, NH₂, NHC(O)CH₃, NHSO₂CH₃, NHCO₂CH₃, tetrazolyl, C(O)piperidin-1-yl, C(O)morpholin-4-yl, C(O)thiomorpholin-4-yl, C(O)-4-methylpiperazin-1-yl, C(O)NHCH₂phenyl, CH₂NHCONH₂, CH₂NHS)₂phenyl, triazolyl, thiadiazolyl, thiazolyl, oxazolyl, pyrazolyl, isoxazolyl, C(O)NH-thiazol-2-yl, C(O)NH-pyrazol-3-yl, or C(O)NHC(CH₃)₃; and
R⁴ is selected from hydrogen, methyl, ethyl, propyl, i-propyl, cyclopropyl, CF₃, phenyl, NH₂, CH₂phenyl, or N(CH₃)CH₂phenyl.

4. The compound according to claim 2, wherein:
R² and R¹ are taken together to form an optionally substituted cyclopento, cyclohexo, cyclohepto, benzo, pyrido, pyridazo, oxacyclohepto, tetrahydroazepino, or thiacyclohepto ring;
R³ is selected from hydrogen, CN, CO₂H, CH₂CN, methyl, CH₂CONH₂, CH₂CO₂CH₃, —C≡CH, C(O)CH₃, CH₂CH₂CN, CH₂CH₂CH₂NH₂, hydrogen, CH₂CO₂H, CO₂Et, CH₂SO₂CH₃, CH₂NHSO₂CH₃, C(O)NH₂, CH₂NHC(O)CH₃, CH₂CH₂OH, C(O)CH₂CH₃, oxadiazolyl, NH₂, NHC(O)CH₃, NHSO₂CH₃, NHCO₂CH₃, tetrazolyl, C(O)piperidin-1-yl, C(O)morpholin-4-yl, C(O)thiomorpholin-4-yl, C(O)-4-methylpiperazin-1-yl, C(O)NHCH₂phenyl, CH₂NHCONH₂, CH₂NHS)₂phenyl, triazolyl, thiadiazolyl, thiazolyl, oxazolyl, pyrazolyl, isoxazolyl, C(O)NH-thiazol-2-yl, C(O)NH-pyrazol-3-yl, or C(O)NHC(CH₃)₃; and
R⁴ is selected from hydrogen, methyl, ethyl, propyl, i-propyl, cyclopropyl, CF₃, phenyl, NH₂, CH₂phenyl, or N(CH₃)CH₂phenyl.

5. The compound according to claim 1, wherein said compound is of formula II-A: 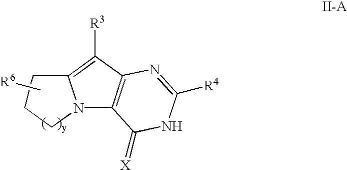
or a pharmaceutically acceptable derivative thereof, wherein:
X is oxygen or sulfur;
y is 0-4;
R³ is selected from R, CN, or Q₍ₙ₎R⁵;
each R is independently selected from hydrogen or an optionally substituted C₁₋₆ aliphatic group;
n is zero or one;
Q is selected from a C₁₋₄ alkylidene chain wherein one methylene unit of Q is optionally replaced by O, S, NR, C(O), CO₂, CONR, NRC(O), NRC(O)NR, SO₂, or NRSO₂;
R⁴ is selected from R, N(R)₂, or an optionally substituted 5-6 membered saturated, partially unsaturated, or fully unsaturated ring having 0-2 heteroatoms independently selected from nitrogen, oxygen, or sulfur;
R⁵ is selected from R or an optionally substituted 5-7 membered saturated, partially unsaturated, or fully unsaturated ring having 0-4 heteroatoms independently selected from nitrogen, oxygen, or sulfur; and
R⁶ is selected from R, OR, N(R)₂, oxo, halogen, NRCO₂R, or NRC(O)R.

6. The compound according to claim 5, wherein:
y is 1-4;
R³ is selected from hydrogen, CN, CO₂H, CH₂CN, methyl, CH₂CONH₂, CH₂CO₂CH₃, —C≡CH, C(O)CH₃, CH₂CH₂CN, CH₂CH₂CH₂NH₂, hydrogen, CH₂CO₂H, CO₂Et, CH₂SO₂CH₃, CH₂NHSO₂CH₃, C(O)NH₂, CH₂NHC(O)CH₃, CH₂CH₂OH, C(O)CH₂CH₃, oxadiazolyl, NH₂, NHC(O)CH₃, NHSO₂CH₃, NHCO₂CH₃, tetrazolyl, C(O)piperidin-1-yl, C(O)morpholin-4-yl, C(O)thiomorpholin-4-yl, C(O)-4-methylpiperazin-1-yl, C(O)NHCH₂phenyl, CH₂NHCONH₂, CH₂NHS)₂phenyl, triazolyl, thiadiazolyl, thiazolyl, oxazolyl, pyrazolyl, isoxazolyl, C(O)NH-thiazol-2-yl, C(O)NH-pyrazol-3-yl, or C(O)NHC(CH₃)₃;
R⁴ is selected from hydrogen, methyl, ethyl, propyl, i-propyl, cyclopropyl, CF₃, phenyl, NH₂, CH₂phenyl, or N(CH₃)CH₂phenyl; and
R⁶ is selected from hydrogen, NH₂, methyl, OCH₃, NHCOCH₃, NHCO₂CH₃, or N(Me)₂

7. The compound according to claim 1, wherein said compound is of formula II-D: 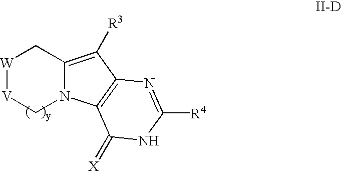
or a pharmaceutically acceptable derivative thereof, wherein:
X is oxygen or sulfur;
y is 1-3;
W—V is selected from CH₂—NH, CH₂—O, CH₂—S, NH—CH₂, O—CH₂, S—CH₂, N═CH, or CH═N;
R³ is selected from R, CN, or Q₍ₙ₎R⁵, wherein n is zero or one;
each R is independently selected from hydrogen or an optionally substituted C₁₋₆ aliphatic group;
Q is selected from a C₁₋₄ alkylidene chain wherein one methylene unit of Q is optionally replaced by O, S, NR, C(O), CO₂, CONR, NRC(O), NRC(O)NR, SO₂, or NRSO₂;
R⁴ is selected from R, N(R)₂, or an optionally substituted 5-6 membered saturated, partially unsaturated, or fully unsaturated ring having 0-2 heteroatoms independently selected from nitrogen, oxygen, or sulfur; and
R⁵ is selected from R or an optionally substituted 5-7 membered saturated, partially unsaturated, or fully unsaturated ring having 0-4 heteroatoms independently selected from nitrogen, oxygen, or sulfur.

8. The compound according to claim 7, wherein:
R³ is selected from hydrogen, CN, CO₂H, CH₂CN, methyl, CH₂CONH₂, CH₂CO₂CH₃, —C≡CH, C(O)CH₃, CH₂CH₂CN, CH₂CH₂CH₂NH₂, hydrogen, CH₂CO₂H, CO₂Et, CH₂SO₂CH₃, CH₂NHSO₂CH₃, C(O)NH₂, CH₂NHC(O)CH₃, CH₂CH₂OH, C(O)CH₂CH₃, oxadiazolyl, NH₂, NHC(O)CH₃, NHSO₂CH₃, NHCO₂CH₃, tetrazolyl, C(O)piperidin-1-yl, C(O)morpholin-4-yl, C(O)thiomorpholin-4-yl, C(O)-4-methylpiperazin-1-yl, C(O)NHCH₂phenyl, CH₂NHCONH₂, CH₂NHS)₂phenyl, triazolyl, thiadiazolyl, thiazolyl, oxazolyl, pyrazolyl, isoxazolyl, C(O)NH-thiazol-2-yl, C(O)NH-pyrazol-3-yl, or C(O)NHC(CH₃)₃; and
R⁴ is selected from hydrogen, methyl, ethyl, propyl, i-propyl, cyclopropyl, CF₃, phenyl, NH₂, CH₂phenyl, or N(CH₃)CH₂phenyl.

9. The compound according to claim 1, wherein:
Y is —S—, and said compound has one or more features selected from the group consisting of:
(a) R² is selected from R, N(R)₂, OR, SR, C(O)R, CO₂R, C(O)N(R)₂, NRN(R)₂, NRC(O)R, SO₂R, or an optionally substituted 5-7 membered saturated, partially unsaturated, or fully unsaturated ring having 0-2 heteroatoms independently selected from nitrogen, oxygen, or sulfur, or R² and R¹ are taken together to form an optionally substituted 5-8 membered saturated, partially unsaturated, or aromatic ring having 0-1 heteroatoms, in addition to the nitrogen of R¹, independently selected from nitrogen, oxygen, or sulfur;
(b) R³ is selected from R, CN, or Q₍ₙ₎R⁵, wherein n is zero or one, Q is selected from a C₁₋₄ alkylidene chain wherein one methylene unit of Q is optionally replaced by O, S, NR, C(O), CO₂, CONR, NRC(O), NRC(O)NR, SO₂, or NRSO₂, and R⁵ is selected from R or an optionally substituted 5-7 membered saturated, partially unsaturated, or fully unsaturated ring having 0-4 heteroatoms independently selected from nitrogen, oxygen, or sulfur; and
(c) R⁴ is selected from R, N(R)₂, or an optionally substituted 5-6 membered saturated, partially unsaturated, or fully unsaturated ring having 0-2 heteroatoms independently selected from nitrogen, oxygen, or sulfur.

10. The compound according to claim 9, wherein:
R² is selected from hydrogen, methyl, ethyl, i-propyl, i-butyl, CF₃, phenyl, CH₂CH₂NH₂, NH₂, NHC(O)CH₃, CH₂CH₂NHC(O)OCH₂phenyl, SCH₃, SO₂CH₃, NHCH₃, SEt, CH₂phenyl, Oi-propyl, morpholin-4-yl, piperidin-1-yl, 4-methyl-piperazin-1-yl, thiomorpholin-4-yl, pyrrolidin-1-yl, thiazol-3-yl, oxazol-3-yl, azepan-1-yl, N(Me)₂, NHi-propyl, NHpropyl, NHi-butyl, NH-cyclopentyl, NH-cyclohexyl, NHCH₂phenyl, NHSO₂CH₃, NHNH₂, N(Me)propyl, NH-cyclopropyl, NHCH₂cyclohexyl, NHCH₂CH₂CH(CH₃)₂, or NHCH₂CH₂imidazol-4-yl;
R³ is selected from hydrogen, CN, CO₂H, CH₂CN, methyl, CH₂CONH₂, CH₂CO₂CH₃, —C≡CH, C(O)CH₃, CH₂CH₂CN, CH₂CH₂CH₂NH₂, hydrogen, CH₂CO₂H, CO₂Et, CH₂SO₂CH₃, CH₂NHSO₂CH₃, C(O)NH₂, CH₂NHC(O)CH₃, CH₂CH₂OH, C(O)CH₂CH₃, oxadiazolyl, NH₂, NHC(O)CH₃, NHSO₂CH₃, NHCO₂CH₃, tetrazolyl, C(O)piperidin-1-yl, C(O)morpholin-4-yl, C(O)thiomorpholin-4-yl, C(O)-4-methylpiperazin-1-yl, C(O)NHCH₂phenyl, CH₂NHCONH₂, CH₂NHS)₂phenyl, triazolyl, thiadiazolyl, thiazolyl, oxazolyl, pyrazolyl, isoxazolyl, C(O)NH-thiazol-2-yl, C(O)NH-pyrazol-3-yl, or C(O)NHC(CH₃)₃; and
R⁴ is selected from hydrogen, methyl, ethyl, propyl, i-propyl, cyclopropyl, CF₃, phenyl, NH₂, CH₂phenyl, or N(CH₃)CH₂phenyl.

11. A compound of formula IV: 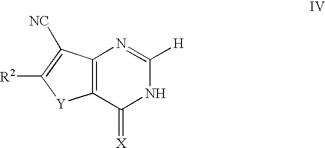
or a pharmaceutically acceptable derivative thereof, wherein:
X is oxygen or sulfur;
Y is —S— or —NR¹—;
R¹ is selected from R, CO₂R, C(O)R, CON(R)₂, SO₂R, SO₂N(R)₂, or an optionally substituted 5-7 membered monocyclic or 8-10 membered bicyclic saturated, partially unsaturated, or fully unsaturated ring having 0-3 heteroatoms independently selected from nitrogen, oxygen, or sulfur;
each R is independently selected from hydrogen or an optionally substituted C₁₋₆ aliphatic group;
R² is selected from R, N(R)₂, OR, SR, C(O)R, CO₂R, C(O)N(R)₂, NRN(R)₂, NRCOR, NRCO₂(C₁₋₆ aliphatic), NRSO₂(C₁₋₆ aliphatic), S(O)(C₁₋₆ aliphatic), SO₂R, SO₂N(R)₂, or an optionally substituted 5-7 membered monocyclic or 8-10 membered bicyclic saturated, partially unsaturated, or fully unsaturated ring having 0-3 heteroatoms independently selected from nitrogen, oxygen, or sulfur, or:
when Y is —NR¹—, R¹ and R² are taken together to form a saturated, partially unsaturated, or fully unsaturated 4-9 membered mono- or bicyclic ring having 1-2 heteroatoms, in addition to the —NR¹— nitrogen, independently selected from nitrogen, oxygen, or sulfur, wherein said ring formed by R¹ and R² is optionally substituted with 1-2 R⁶; or
R⁵ is selected from R or an optionally substituted 5-14 membered mono-, bi-, or tricyclic aromatic, partially unsaturated, or saturated ring having 0-4 heteroatoms independently selected from nitrogen, oxygen, or sulfur; and
each R⁶ is independently selected from R, oxo, halogen, CN, C(O)R, CO₂R, SO₂R, OR, SR, N(R)₂, NRC(O)R, C(O)N(R)₂, NRCO₂R, OC(O)N(R)₂, NRSO₂R, or SO₂NR.

12. The compound according to claim 11, wherein:
Y is —NR¹—.

13. The compound according to claim 11, wherein:
Y is —S—.

14. The compound according to claim 5, wherein said compound is selected from any one of the following compounds of formula II-A:

| | |
|---|---|
| | II-A |
| 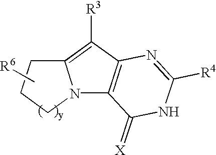 | |
| No. | y | X | R³ | R⁴ | R⁶ |
|---|---|---|---|---|---|
| II-A1 | 1 | S | —CN | H | H |
| II-A2 | 2 | S | —CN | H | H |
| II-A3 | 3 | S | —CN | H | H |
| II-A4 | 4 | S | —CN | H | H |
| II-A5 | 3 | S | —CO₂H | H | H |
| II-A6 | 3 | S | —CH₂CN | H | H |
| II-A7 | 3 | S | —CH₃ | H | H |
| II-A8 | 3 | S | —CH₂CONH₂ | H | H |
| II-A9 | 3 | S | —CH₂CO₂CH₃ | H | H |
| II-A10 | 3 | S | —C≡CH | H | H |
| II-A11 | 3 | S | —COCH₃ | H | H |
| II-A12 | 3 | S | —C(CH₃)═N—OCH₃ | H | H |
| II-A13 | 3 | S | —CH₂CH₂CN | H | H |
| II-A14 | 3 | S | —C(CH₃)═NNHCH₃ | H | H |
| II-A15 | 3 | S | —CH₂CH₂CH₂NH₂ | H | H |
| II-A16 | 3 | S | —CN | H | H |
| II-A17 | 3 | S | —H | H | H |
| II-A18 | 3 | S | —CN | H | H |
| II-A19 | 3 | S | —CH₂CO₂H | H | H |
| II-A20 | 3 | S | —CO₂CH₂CH₃ | H | H |
| II-A21 | 3 | S | —CH₂SO₂CH₃ | H | H |
| II-A22 | 3 | S | —CH₂NHSO₂CH₃ | H | H |
| II-A23 | 3 | S | —CH₂NHCOCH₃ | H | H |
| II-A24 | 3 | S | —CH₂CH₂OH | H | H |
| II-A25 | 3 | S | —COCH₂CH₃ | H | H |
| II-A26 | 3 | S | 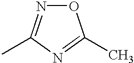 | H | H |
| II-A27 | 3 | S | 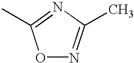 | H | H |
| II-A28 | 3 | S | 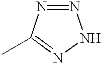 | H | H |
| II-A29 | 3 | S | 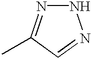 | H | H |
| II-A30 | 3 | S | 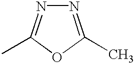 | H | H |
| II-A31 | 3 | S | 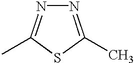 | H | H |
| II-A32 | 3 | S | 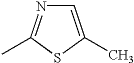 | H | H |
| II-A33 | 3 | S | 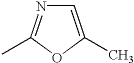 | H | H |
| II-A34 | 3 | S | 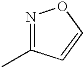 | H | H |
| II-A35 | 3 | S | 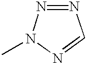 | H | H |
| II-A36 | 3 | S | 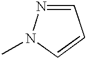 | H | H |
| II-A37 | 3 | S | 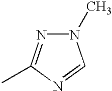 | H | H |
| II-A38 | 3 | S | 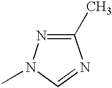 | H | H |
| II-A39 | 3 | S | 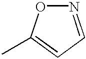 | H | H |
| II-A40 | 3 | S | 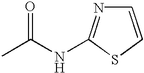 | H | H |
| II-A41 | 3 | S | 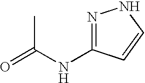 | H | H |
| II-A42 | 3 | S | 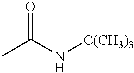 | H | H |
| II-A43 | 3 | S | 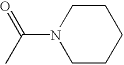 | H | H |
| II-A44 | 3 | S | 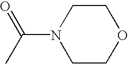 | H | H |
| II-A45 | 3 | S | 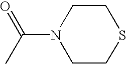 | H | H |
| II-A46 | 3 | S | 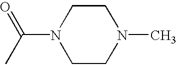 | H | H |
| II-A47 | 3 | S | 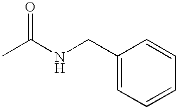 | H | H |
| II-A48 | 3 | S | —CH₂NHCONH₂ | H | H |
| II-A49 | 3 | S | 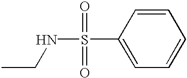 | H | H |
| II-A50 | 3 | S | —CN | H | 9-NH₂ |
| II-A51 | 3 | S | —CN | H | 9-NHCOCH₃ |
| II-A52 | 3 | S | —CN | H | 8-NH₂ |
| II-A53 | 3 | S | —CN | H | 8-NHCOCH₃ |
| II-A54 | 3 | S | —CN | H | 9-CH₃ |
| II-A55 | 3 | S | —CN | H | 8-OCH₃ |
| II-A56 | 3 | S | —CN | H | 8,9-Me₂ |
| II-A57 | 3 | S | —CN | H | 8-NHCO₂Me |
| II-A58 | 3 | S | —CN | H | 8-NMe₂ |
| II-A59 | 3 | S | —CN | CH₃ | H |
| II-A60 | 3 | S | —CN | CF₃ | H |
| II-A61 | 3 | S | —CN | Pr | H |
| II-A62 | 3 | S | —CN | Ph | H |
| II-A63 | 3 | S | —CN | CHMe₂ | H |
| II-A64 | 3 | S | —CN | NH₂ | H |
| II-A65 | 3 | S | —CN | CH₃ | H |
| II-A66 | 2 | S | —CN | CF₃ | H |
| II-A67 | 3 | S | —CN | CH₂Ph | H |
| II-A68 | 3 | O | —CN | H | H |
| II-A69 | 2 | O | —CN | H | H |
| II-A70 | 3 | O | —CN | CH₃ | H |
| II-A71 | 3 | O | —CN | cyclo-Pr | H |
| II-A72 | 3 | O | —CN | N(Me)CH₂Ph | H |
| II-A73 | 3 | O | —CO₂H | H | H |
| II-A74 | 3 | O | —CONH₂ | H | H |
| II-A75 | 3 | O | —H | H | H |
| II-A76 | 4 | O | —CN | H | H |
| II-A77 | 3 | S | —NH₂ | H | H |
| II-A78 | 3 | S | —NHR | H | H |
| II-A79 | 3 | S | —NHAc | H | H |
| II-A80 | 3 | S | —NHSO₂R | H | H |
| II-A81 | 3 | S | —NHCO₂R | H | H |
| II-A82 | 3 | S | —CONH₂ | H | H. |

15. The compound according to claim 2, wherein said compound is selected from any one of the following compounds of formula II-B:

| | | | | | II-B |
|---|---|---|---|---|---|
| 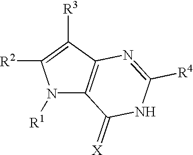 | | | | | |
| No. | X | R¹ | R² | R³ | R⁴ |
| II-B1 | O | Et | Et | CN | H |
| II-B2 | S | Et | Et | CN | H |
| II-B3 | S | H | Et | CN | H |
| II-B4 | S | Ph | Et | CN | H |
| II-B5 | S | CH₂CH₂(morpholin- | Et | CN | H |
| | | 4-yl) | | | |
| II-B6 | S | isobutyl | isobutyl | CN | H |
| II-B7 | S | isobutyl | CF₃ | CN | H |
| II-B8 | S | CH₂Ph | CF₃ | CN | H |
| II-B9 | S | CH₂CH₂(morpholin- | CF₃ | CN | H |
| | | 4-yl) | | | |
| II-B10 | O | Ph | Me | CN | H |
| II-B11 | S | Ph | Me | CN | H |
| II-B12 | O | Ph | H | CN | H |
| II-B13 | S | Ph | H | CN | H |
| II-B14 | O | Et | Et | CN | H |
| II-B15 | O | H | Et | CN | H |
| II-B16 | S | CH₂CH₂Ph | Et | CN | H |
| II-B17 | O | Ph | Ph | CN | H |
| II-B18 | S | Ph | Ph | CN | H |
| II-B19 | S | COCH₃ | Et | CN | H |
| II-B20 | S | CONH₂ | Et | CN | H |
| II-B21 | S | CH₂CONH₂ | Et | CN | H |
| II-B22 | S | SO₂CH₃ | Et | CN | H |
| II-B23 | S | CH₂SO₂NH₂ | Et | CN | H |
| II-B24 | S | CO₂Et | Et | CN | H |
| II-B25 | S | cyclopropyl | Et | CN | H |
| II-B26 | S | Et | Ph | CN | H |
| II-B27 | O | Et | CH₂CH₂NH₂ | CN | H |
| II-B28 | S | isopropyl | isopropyl | CN | H |
| II-B29 | O | isobutyl | isobutyl | CN | H |
| II-B30 | O | Et | CH₂CH₂NHCbz | CN | H |
| II-B31 | S | Et | CH₂CH₂NHCbz | CN | H |
| II-B32 | O | Et | Ph | CN | H. |

16. The compound according to claim 7, wherein said compound is selected from any one of the following compounds of formula II-D:

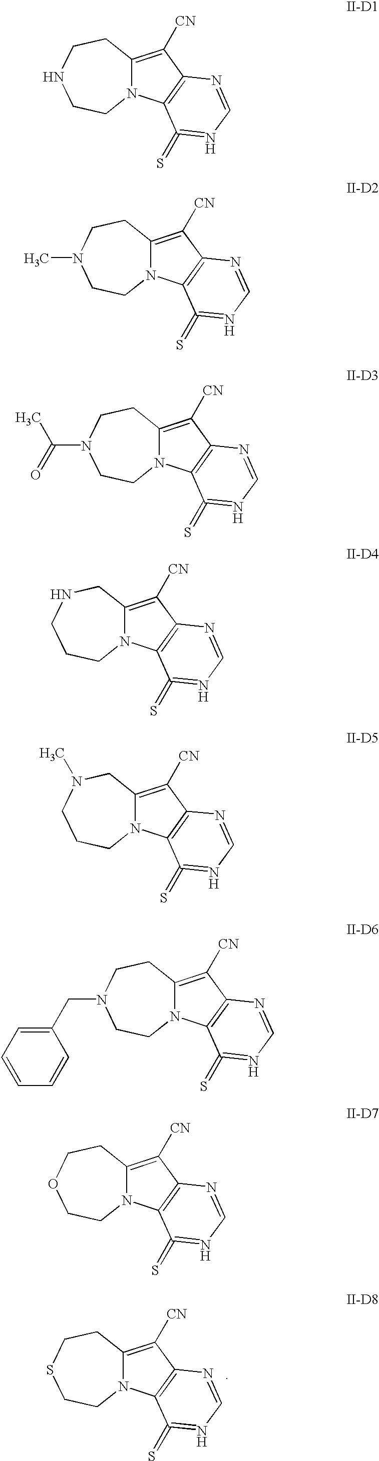

17. The compound according to claim 9, wherein said compound is selected from any one of the following compounds of formula III:

| | |
|---|---|
| | III |
| 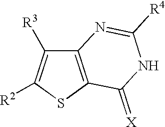 | |
| No. | X | R² | R³ | R⁴ |
|---|---|---|---|---|
| III-1 | S | H | CN | H |
| III-2 | S | NH₂ | CN | H |
| III-3 | S | NHCOCH₃ | CN | H |
| III-4 | O | SCH₃ | CN | H |
| III-5 | S | SCH₃ | CN | H |
| III-6 | S | SO₂CH₃ | CN | H |
| III-7 | S | NHCH₃ | CN | H |
| III-8 | S | SCH₂CH₃ | CN | H |
| III-9 | S | CH₂Ph | CN | H |
| III-10 | S | OCH(CH₃)₂ | CN | H |
| III-11 | S | CH₂CH₃ | CN | H |
| III-12 | S | 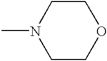 | CN | H |
| III-13 | S | 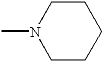 | CN | H |
| III-14 | S | 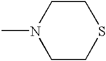 | CN | H |
| III-15 | S | 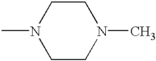 | CN | H |
| III-16 | S | 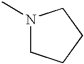 | CN | H |
| III-17 | S | 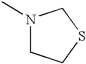 | CN | H |
| III-18 | S | 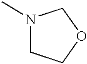 | CN | H |
| III-19 | S | 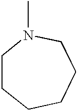 | CN | H |
| III-20 | S | N(Me)₂ | CN | H |
| III-21 | O | NHCH(CH₃)₂ | CN | H |
| III-22 | O | NHCH₂CH₂CH₃ | CN | H |
| III-23 | O | NHCH₂CH(CH₃)₂ | CN | H |
| III-24 | O | 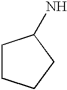 | CN | H |
| III-25 | O | 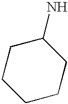 | CN | H |
| III-26 | O | NHCH₂Ph | CN | H |
| III-27 | S | NHSO₂R | CN | H |
| III-28 | O | NH₂ | CN | H |
| III-30 | O | NHCH(CH₃)₂ | C(═NH)NHCH(CH₃)₂ | H |
| III-31 | O | NHCH₂CH(CH₃)₂ | C(═NH)NHCH(CH₃)₂ | H |
| III-32 | O | NHNH₂ | CN | H |
| III-33 | O | 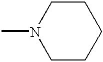 | CN | H |
| III-34 | O | 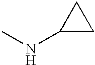 | CN | H |
| III-35 | O | 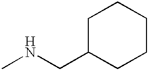 | CN | H |
| III-36 | O | NHCH₂CH₂CH(CH₃)₂ | CN | H |
| III-37 | O | 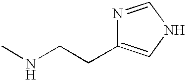 | CN | H |
| III-38 | O | CH₂CH₃ | CN | H |
| III-39 | O | N(CH₃)CH₂CH₂CH₃ | CN | H. |

18. A composition comprising a compound according to claim 1, and a pharmaceutically acceptable carrier, adjuvant, or vehicle.

19. The composition according to claim 18, additionally comprising an additional therapeutic agent selected from:
(a) a neurotrophic factor; or
(b) an agent for treating diabetes.

20. A method of inhibiting GSK-3 kinase activity in a biological sample comprising the step of contacting said biological sample with:
a) a compound according to claim 1; or
b) a composition according to claim 18.

21. A method of treating or lessening the severity of a GSK-3-mediated disease or condition in a patient comprising the step of administering to said patient a composition according to claim 18.

22. A method of treating or lessening the severity of a disease or condition in a patient selected from diabetes, a neurodegenerative disease, AIDS associated dementia, multiple sclerosis (MS), schizophrenia, cardiomycete hypertrophy, or baldness, comprising the step of administering to said patient a composition according to claim 18.

23. The method according to claim 21, comprising the additional step of administering to said patient an additional therapeutic agent, wherein:
said additional therapeutic agent is appropriate for the disease being treated; and
said additional therapeutic agent is administered together with said composition as a single dosage form or separately from said composition as part of a multiple dosage form.

24. A method of inhibiting ROCK kinase activity in a biological sample comprising the step of contacting said biological sample with:
(a) a compound according to claim 9; or
(b) a composition comprising a compound according to claim 9, and a pharmaceutically acceptable carrier, adjuvant, or vehicle.

25. A method of treating or lessening the severity of a ROCK-mediated disease or condition in a patient comprising the step of administering to said patient a composition comprising a compound according to claim 9, and a pharmaceutically acceptable carrier, adjuvant, or vehicle.

26. A method of treating or lessening the severity of a disease or condition in a patient selected from hypertension, erectile dysfunction, angiogenesis, neuroregeneration, metastasis, glaucoma, inflammation, artheriosclerosis, immunosuppresion, restenosis, asthma, or cardiac hypertrophy, comprising the step of administering to said patient a composition comprising a compound according to claim 9, and a pharmaceutically acceptable carrier, adjuvant, or vehicle.

27. The method according to claim 26, comprising the additional step of administering to said patient an additional therapeutic agent, wherein:
said additional therapeutic agent is appropriate for the disease being treated; and
said additional therapeutic agent is administered together with said composition as a single dosage form or separately from said composition as part of a multiple dosage form.

28. A composition for coating an implantable device comprising a compound according to claim 1 and a carrier suitable for coating said implantable device.

29. An implantable device coated with a composition according to claim 28.
